(19) United States
(12) Patent Application Publication
(10) Pub. No.: US 2026/0263383 A1
(43) Pub. Date: Sep. 10, 2026

(54) TRANSDERMAL DRUG DELIVERY PATCH SYSTEM FOR CONTROLLED AND SUSTAINED DELIVERY OF TIZANIDINE BASE

(71) Applicant: Christine RITTENHOUSE, Collegeville, PA (US)

(72) Inventor: Christine RITTENHOUSE, Collegeville, PA (US)

(21) Appl. No.: 19/319,422

(22) Filed: Sep. 4, 2025

Related U.S. Application Data
(60) Provisional application No. 63/690,629, filed on Sep. 4, 2024.

(51) Int. Cl. A61K 9/70; A61K 31/431; A61K 47/10; A61K 47/22; A61P 21/02

(52) U.S. Cl. CPC A61K 9/7061; A61K 31/431; A61K 47/10; A61K 47/22; A61P 21/02

(57) ABSTRACT

A transdermal drug delivery patch system for controlled and sustained delivery of tizanidine base. The patch includes an adhesive matrix containing a pressure-sensitive adhesive and tizanidine base, dispersed together with at least one permeation enhancer. The patch includes a release liner and a backing film. Configured for titratable dosing, each patch delivers tizanidine base transdermally over a period of time, achieving content uniformity, process controls, and impurity limits suitable for commercial use. The delivery system provides steady-state plasma levels, reduced Cmax, minimized gastrointestinal side effects, and bypasses first-pass metabolism, offering improved therapy options for patients intolerant or unable to take oral muscle relaxants. Kits and methods for individualized dosing and safe application in clinical or home settings are provided.

### CROSS-REFERENCE TO RELATED APPLICATIONS

This application claims the benefit of priority to U.S. Provisional Application No. 63/690,629, filed Sep. 4, 2024, the contents of which is hereby incorporated by reference herein.

### BACKGROUND

Current therapeutic management of muscle spasticity frequently relies on pharmacological agents such as tizanidine, a centrally acting alpha-2 adrenergic agonist. Tizanidine is typically administered in oral tablet or capsule form, modalities that are associated with several well-documented challenges in clinical practice. Oral delivery commonly results in significant pharmacokinetic variability-including pronounced fluctuations in drug plasma concentrations due to hepatic first-pass metabolism and variable absorption in the fed versus fasted state. These fluctuations often lead to peak-related side effects such as hypotension, sedation, and substantial gastrointestinal disturbances, including abdominal pain and diarrhea. Furthermore, oral tizanidine products may be poorly tolerated or unsuitable for patients who experience dysphagia, gastrointestinal intolerance, or who, due to underlying conditions or age, are unable or unwilling to swallow solid medications.

While transdermal drug delivery systems have been developed for a range of therapeutic agents-such as fentanyl, clonidine, and nicotine-adaptation for muscle relaxants like tizanidine has posed significant technical hurdles. Existing transdermal formulations for other active ingredients have encountered difficulties in delivering poorly soluble compounds over extended durations while maintaining consistent drug release and patch adhesion, achieving adequate skin permeation, and ensuring stability and content uniformity. Attempts at transdermal delivery for muscle relaxants or related drugs are frequently limited by inadequate skin absorption, poor control over the rate and extent of drug release, or instability of the active compound within the adhesive or matrix. There remain substantial unmet needs for improved delivery platforms that can provide controlled, sustained, and titratable administration of muscle relaxants and centrally acting antispasmodics, particularly in patient populations who are not well-served by conventional oral dosage forms.

### SUMMARY

The present invention provides for a transdermal drug delivery patch includes (1) an adhesive matrix containing a pressure-sensitive adhesive; (2) tizanidine base as the active pharmaceutical ingredient, the tizanidine base dispersed within the adhesive matrix; (3) at least one permeation enhancer; and (4) solvent system.

The present invention also provides for a transdermal drug delivery patch that includes: a monolithic adhesive matrix including an acrylic pressure-sensitive adhesive, dispersed within the adhesive matrix, an effective amount of finely ground, micronized, or milled tizanidine base as the active pharmaceutical ingredient, at least one permeation enhancer selected from povidone and diethylene glycol, a solvent system including ethanol and/or ethyl acetate present during preparation of the adhesive matrix, the monolithic adhesive matrix having a thickness of 0.013±0.004 inches, a release liner including a fluoropolymer-coated polyester film in contact with the adhesive matrix, and a backing film including a laminate including at least one polymeric film and a barrier layer, wherein the patch has an area between 5 cm² and 20 cm² and is adapted to release tizanidine base transdermally over a period of at least 24 to 48 hours.

The present invention also provides a method of transdermally administering tizanidine base by applying to the skin the transdermal drug delivery patch described herein. The method is carried out to treat a disease or disorder ameliorated by tizanidine. Such diseases or disorders ameliorated by tizanidine include, for example, FDA-approved indications for the management of muscle spasticity associated with conditions such as multiple sclerosis, spinal cord injury, or acquired brain injury. Such diseases or disorders ameliorated by tizanidine also include, for example, off-label indications for treating chronic neck and lower back pain, regional musculoskeletal pain syndromes, migraine (including analgesic rebound or medication-overuse headache), fibromyalgia, refractory insomnia in spastic quadriplegic patients, use as an adjunct during opioid tapering, and occasional use as part of an anticonvulsant regimen for seizure control.

The present invention also provides a method of treating increased muscle tone associated with spasticity in a subject in need thereof. The method includes transdermally administering tizanidine base by applying to the skin the transdermal drug delivery patch described herein, wherein said patch is adapted to deliver an effective amount of tizanidine base over a period of at least 24 to 48 hours to reduce muscle tone and/or alleviate the symptoms of spasticity, in patients who are unable or unwilling to swallow oral medication, whom oral therapy is not well tolerated, or who have demonstrated intolerance to oral tizanidine therapy.

The present invention also provides a kit includes: at least one transdermal patch described herein; separately packaged with the at least one patch, instructions for use directing application of the patch to the skin to treat increased muscle tone associated with spasticity, and provide titratable dosing; and further including at least one additional component selected from: (a) individually sealed pouches for each patch, (b) a child-resistant package, (c) a calendar or dosing schedule aid, (d) a disposal pouch for used patches, (e) a titration card or dose adjustment guide, (f) a desiccant or moisture-absorbing element, (g) a temperature indicator, (h) a QR code or web address for digital instructions, (i) multilingual instructions, and (j) a sterile wipe or pad for skin preparation.

The present invention also provides a kit for transdermal administration of tizanidine base. The kit includes: at least one transdermal patch described herein; wherein the patch, when applied as directed, is configured to deliver tizanidine base with a reduced incidence of gastrointestinal side effects and with steady-state plasma levels compared to oral administration; and, separately packaged with the at least one patch, instructions for use directing application of the patch to the skin to treat increased muscle tone associated with spasticity, provide titratable dosing, and maintain reduced gastrointestinal side effects relative to oral tizanidine therapy; and further including at least one additional component selected from: (a) individually sealed pouches for each patch, (b) a child-resistant package, (c) a calendar or dosing schedule aid, (d) a disposal pouch for used patches, (e) a titration card or dose adjustment guide, (f) a desiccant or moisture-absorbing element, (g) a temperature indicator, (h) a QR code or web address for digital instructions, (i) multilingual instructions, and (j) a sterile wipe or pad for skin preparation.

The present invention also provides a method of topically applying to the skin transdermal patch described herein, to deliver an effective amount of tizanidine base, to reduce muscle tone and/or alleviates the symptoms of spasticity.

The present invention confers numerous advantages over prior art transdermal and oral drug delivery products, particularly addressing unmet clinical and technical challenges associated with the administration of tizanidine and similar muscle relaxants. Unlike conventional oral dosage forms, which suffer from pronounced pharmacokinetic variability due to hepatic first-pass metabolism and fluctuating absorption depending on fed or fasted states, the described transdermal patch achieves controlled, sustained, and titratable systemic delivery of tizanidine base over extended intervals of at least 24 to 48 hours. This unique pharmacokinetic profile eliminates oral dose-related spikes in plasma concentration, markedly reduces gastrointestinal side effects such as abdominal pain and diarrhea, and provides a viable option for patients unable or unwilling to swallow oral medications. The patch's use of a monolithic acrylic adhesive matrix, combined with finely micronized tizanidine base and carefully selected permeation enhancers, ensures content uniformity (±5%) and batch-to-batch reproducibility, supporting both individualized titration and robust manufacturing at scale. Further, the use of an occlusive, multi-layer backing film and high-performance release liner assures optimal adhesion, environmental protection, and ease of handling, distinguishing the system from earlier transdermal technologies that struggled with inadequate skin permeation, instability, or cumbersome application.

Surprisingly and unexpectedly, the present invention demonstrated in both in vitro and in vivo studies that transdermal delivery of tizanidine base could achieve plasma exposure (AUC) bioequivalent to oral dosing, while maintaining a substantially lower maximum plasma concentration (Cmax), thereby minimizing the risk of dose-related adverse effects and drug interactions. Comparative pharmacokinetic studies revealed that the patch provided highly consistent drug absorption regardless of feeding state, in stark contrast to the significant variability observed with oral tablets and capsules. Notably, the patch system delivered linear and sustained drug release even with the poor aqueous solubility of tizanidine base, a feat previously considered technically challenging, if not prohibitive, in the field of muscle relaxant therapy. The rigorous solvent, adhesive, and enhancer screening undertaken in the present invention resulted in a robust formulation that remained chemically stable over extended storage, with impurity levels reliably maintained below 0.1% for at least 12 months—another outcome not anticipated based on prior art products or earlier attempts at transdermal muscle relaxant delivery.

Secondary considerations further underscore the unobvious and nontrivial nature of the present invention. The patch was shown to improve patient adherence due to once-daily or twice-weekly administration, compared to multiple daily oral doses, and was particularly well-tolerated by populations prone to gastrointestinal intolerance, dysphagia, or variable oral absorption. The inclusion of titratable patch sizes, individually sealed packaging, child-resistant features, and comprehensive kit components—all designed to ensure user safety, correct application, and easy disposal—further enhance the commercial and practical value of the invention. Collectively, these clinical, pharmacological, technical, and patient-centric results demonstrate a marked advance over prior art, providing a novel and unexpectedly effective solution to longstanding problems in the sustained and controlled administration of tizanidine and related therapeutic agents.

### BRIEF DESCRIPTION OF THE FIGURES

FIG. 1A. FIG. 1A illustrates representative high-performance liquid chromatography (HPLC) chromatograms generated during impurity testing of tizanidine base following extraction with various solvents. The figure compares impurity peak profiles for extracts prepared using acetone, ethyl acetate, methanol, tetrahydrofuran (THF), and chloroform, demonstrating the pronounced impurity peak associated with acetone and the minimal or absent impurity formation observed with ethyl acetate, methanol, and other Class 3 solvents. This result underscores the solvent-dependent stability of the active pharmaceutical ingredient in the patch formulations, guiding solvent selection for manufacturing. This figure further highlights that the presence of a significant impurity peak with acetone reinforces its exclusion from all final and commercial patch formulations, ensuring chemical stability and regulatory compliance.

FIG. 1B. FIG. 1B provides additional HPLC impurity profiles for tizanidine patch extracts, corroborating the results shown in FIG. 1A. The data reinforces the finding that acetone exposure during patch preparation induces significant impurity formation, while ethyl acetate and methanol yield low-impurity extracts. Together, FIGS. 1A and 1B support the exclusion of acetone from all manufacturing steps to maintain product stability and compliance with impurity limits.

FIG. 2A. FIG. 2A presents the cumulative in vitro dissolution profile of tizanidine base delivered from the GSP6 patch formulation (5 cm² patch size) over a 72-hour period in neutral pH buffer. The plot demonstrates a linear and sustained release of tizanidine, confirming the formulation's ability to achieve controlled drug delivery consistent with transdermal therapeutic requirements.

FIG. 2B. FIG. 2B shows the cumulative in vitro dissolution profile for the GSP413 patch formulation (5 cm² patch size) under identical test conditions as FIG. 2A. The graph highlights a similar linear and extended release of tizanidine base, validating the performance and reproducibility of the alternative formulation and supporting its use as a backup or supplementary product.

FIG. 3A. FIG. 3A depicts the in vitro skin permeation delivery rate (mg/time) of tizanidine from the GSP6 patch formulation using a 0.71 cm² patch sample, measured at successive time points over 72 hours. The figure captures the rate of transdermal drug flux, illustrating continuous and consistent permeation through the skin model.

FIG. 3B. FIG. 3B displays the cumulative in vitro skin permeation profile (total mg delivered over time) for the GSP6 formulation (0.71 cm² patch). This plot demonstrates robust, time-dependent drug delivery and confirms the ability of the patch to achieve therapeutic exposure over the intended wear period.

FIG. 4A. FIG. 4A illustrates the in vitro skin permeation delivery rate for the GSP413 patch formulation (0.71 cm² patch), plotting the amount of tizanidine delivered per unit time across 72 hours. The data show sustained delivery characteristics, complementing the findings for the GSP6 formulation.

FIG. 4B. FIG. 4B presents the cumulative skin permeation profile (total mg delivered over time) for the GSP413 patch formulation (0.71 cm² patch), confirming that this formulation and supports bioequivalence across patch options.

FIG. 5. FIG. 5 is an exploded schematic view of the tizanidine transdermal patch. It details the assembly and spatial relationship between the major components, including the backing film, drug-in-adhesive monolith, enhancer additives, and release liner. The illustration clarifies the structure of the patch, showing the arrangement necessary for effective drug delivery and robust product construction.

FIG. 6. FIG. 6 provides a top-down plan view of the assembled tizanidine transdermal patch, highlighting the external geometry, patch area, and placement of the scored release liner for user removal. It serves as a visual guide for proper handling and application, demonstrates the uniformity and reproducibility of the final product.

### DETAILED DESCRIPTION

The present invention can be more readily understood by reading the following detailed description of the invention and study of the included examples.

As used herein, the following terms have the meanings ascribed to them unless specified otherwise.

The terms “comprise,” “comprising,” “include,” “including,” and “includes” when used in this specification and claims are intended to specify the presence of stated substances, features, integers, components, or steps, but they do not preclude the presence or addition of one or more other substances, features, integers, components, steps, or combinations thereof.

The term “about” modifies the subject values, such that they are within an acceptable error range, as determined by one of ordinary skill in the art, which will depend in part on the limitations of the measurement system.

The articles “a” and “an” as used herein refers to “one or more” or “at least one,” unless otherwise indicated. That is, reference to any element or component of an embodiment by the indefinite article “a” or “an” does not exclude the possibility that more than one element or component is present.

The term “transdermal drug delivery patch” refers to a medical device configured for application to the skin, including a drug-containing adhesive matrix, a release liner, and a backing film, structured and formulated to deliver an active pharmaceutical ingredient through the skin and into systemic circulation over a defined period.

The term “monolithic adhesive matrix” refers to a continuous, homogeneous layer formed from a pressure-sensitive adhesive in which the active pharmaceutical ingredient and any enhancers or solvents are dispersed throughout the matrix before application, such that no discrete reservoir or separate stratified layer is present.

The term “acrylic pressure-sensitive adhesive” refers to a tacky, viscoelastic polymer based on acrylic copolymers that adheres to skin and substrate surfaces with gentle pressure, without requiring activation by heat, moisture, or solvent, and is suitable for biomedical patch applications.

The term “tizanidine base” refers to the free base form of tizanidine, a centrally acting alpha-2 adrenergic agonist indicated for muscle spasticity, and includes both coarse crystalline and micronized particulate forms suitable for drug delivery matrices. Tizanidine is primarily FDA-approved for the management of muscle spasticity, especially in conditions such as multiple sclerosis and spinal cord injury. However, it is also prescribed for a variety of off-label uses. These include treatment of chronic neck and lower back pain, regional musculoskeletal pain syndromes, migraines (including analgesic rebound headaches), fibromyalgia, and refractory insomnia in spastic quadriplegic patients. In clinical practice, tizanidine may also be used as an adjunct for pain management during opioid tapering and occasionally as part of an anticonvulsant regimen—though such applications are not FDA-approved and rely on clinician judgment and available evidence. While supported by some research and case reports, use in these non-spasticity contexts should be carefully evaluated and monitored, as the safety and long-term efficacy have not been established by regulatory authorities.

The term “micronized” refers to having a particle size less than 30 microns D90 by volume, as measured by laser diffraction, resulting in improved dispersion and dissolution properties relative to larger or unground particles.

The term “permeation enhancer” refers to any additive compound incorporated into the adhesive matrix that increases the rate or extent of transdermal absorption by modifying the barrier properties of skin, including but not limited to polyvinylpyrrolidone and diethylene glycol.

The term “solvent system” refers to the mixture of volatile organic solvents present during preparation of the adhesive matrix, specifically including ethanol and/or ethyl acetate, which facilitate dispersion of the active ingredient, dissolution of excipients, and process compatibility with adhesives.

The term “release liner” refers to a removable layer, typically of fluoropolymer-coated polyester film, which temporarily contacts the adhesive until application and protects the drug matrix from premature exposure or contamination.

The term “backing film” refers to a flexible, non-adhesive layer including at least one polymeric film and optionally a barrier layer, attached to the opposite side of the patch from the release liner, providing support, occlusivity, and protection for the adhesive matrix.

The term “coat weight” refers to the mass of the adhesive matrix per unit area on the patch, typically measured in grams per square centimeter, and excludes the weight of the release liner and backing film.

The term “content uniformity” refers to the consistency of the active pharmaceutical ingredient's distribution within and across individual patches and batches, such that the drug content does not vary by more than a specified percentage, e.g., ±5%.

The term “drug load” refers to the percentage by weight of the active pharmaceutical ingredient relative to total solids present in the adhesive matrix, excluding solvents lost during drying.

The term “impurity” refers to any substance other than the intended active pharmaceutical ingredient, excipients, or components, arising from synthesis, degradation, processing, or storage, and detected and quantified by validated analytical methods such as HPLC.

The term “dissolution profile” refers to the measured release kinetics of the active drug from the patch into an external medium over time, typically characterized by cumulative drug released as determined by in vitro methods simulating physiological conditions.

The term “skin permeation” refers to the passage of the active pharmaceutical ingredient through the skin layers and into systemic circulation, as measured by in vitro diffusion cell assays or in vivo pharmacokinetic studies.

The term “rectangular patch with rounded corners” refers to a patch geometry whose periphery forms a rectangle with its corners smoothly curved rather than sharp, facilitating comfort and wearability.

The term “score line” refers to a physical indentation or marking extending across the release liner, designed to facilitate separation and removal of the liner from the adhesive prior to use.

The term “occlusive backing” refers to a backing film possessing a moisture vapor transmission rate (MVTR) less than, e.g., 1 g/m²/24 hr, capable of limiting water vapor loss from the skin and protecting the matrix from external moisture.

The term “light-impermeable pouch” refers to a packaging unit constructed from materials, such as aluminum laminate, that blocks the transmission of light to prevent photodegradation of the patch contents during storage.

The term “titratable dosing” refers to the ability to incrementally adjust drug delivery by providing patches in multiple discrete sizes or strengths, allowing individualized dosing based on therapeutic response.

The term “steady-state plasma concentration” refers to the condition in which the rate of drug input through the skin equals the rate of elimination, maintaining constant systemic levels of the active ingredient over the intended wear time.

The term “Cmax” refers to the maximum observed plasma concentration of the active pharmaceutical ingredient following administration, as measured in pharmacokinetic studies.

The term “fed/unfed pharmacokinetic variability” refers to differences in systemic drug exposure arising from whether an oral dose is administered with or without food, which is eliminated by transdermal delivery.

The term “patch kit” refers to a product assembly that includes at least one individually pouched patch, instructions for use, and optionally further components such as child-resistant packaging, dosing guides, disposal pouches, desiccants, temperature indicators, and digital support resources.

The term “manufacturing process” refers to the sequence of operations for preparing, coating, drying, laminating, die-cutting, and packaging the transdermal patch, including the specified order of ingredient addition and in-process controls for thickness, weight, and uniformity.

The term “in-process quality control” refers to any analytical, physical, or procedural check performed during the manufacturing sequence to verify that intermediate and final product parameters meet defined specifications for performance and safety.

The term “storage stability” refers to the retention of drug content, impurity profile, and patch integrity when kept under specified ambient conditions for a defined period, such as 12 months, without significant change in efficacy or safety.

The term “pharmacokinetic equivalence” refers to the condition where the systemic exposure produced by the patch matches or approximates the area under the time-concentration curve (AUC) for an equivalent oral dose, enabling similar therapeutic results.

The term “penetration enhancer” refers to any compound capable of increasing the flux of the active pharmaceutical ingredient across the skin when incorporated into the patch matrix, including but not limited to polyvinylpyrrolidone and diethylene glycol.

The term “polyvinylpyrrolidone (povidone)” refers to a synthetic polymer used as a penetration enhancer, with molecular weight grades such as K29/32 or K90.

The term “diethylene glycol (DEG)” refers to a hygroscopic organic compound used as a penetration enhancer in transdermal formulations to improve drug absorption through the skin.

The term “ethylene acetate” refers to a volatile, polar solvent used in patch manufacturing for its compatibility with acrylic adhesives and ability to disperse drug particles in formulation.

The term “ethanol” refers to a volatile alcohol solvent used in patch manufacturing for its compatibility with adhesives, drug solubilization, and process efficacy.

The term “acrylic copolymer with —COOH or —OH functional group” refers to an acrylic-based pressure-sensitive adhesive containing carboxyl (—COOH) or hydroxyl (—OH) functional groups, which impact adhesive properties and compatibility with drug and excipients.

The term “fluoropolymer-coated polyester film” refers to a polyester substrate coated with a fluoropolymer layer, conferring easy-release and non-stick properties essential for use as a release liner.

The term “barrier layer” refers to any film, metallic vapor deposit, or polymer layer in the backing film that restricts the passage of moisture, oxygen, or light, facilitating patch stability and shelf life.

The term “moisture vapor transmission rate (MVTR)” refers to the quantifiable rate at which water vapor passes through a material, with low MVTR indicating higher occlusiveness relevant to patch backing films.

The term “content uniformity” refers to the degree to which drug content is consistent across the entire patch matrix and batch, measured by assaying portions from different regions and compared to the mean; permissible variation typically ±5%.

The term “impurity profile” refers to the identification and quantification of process- or storage-related impurities in the patch matrix using validated analytical techniques, e.g., HPLC, controlled to specified upper limits.

The term “area under the curve (AUC)” refers to the integrated area under the time-concentration curve in pharmacokinetic studies, representing total systemic exposure to the active drug over a given time frame.

The term “kit component” refers to any supplementary item provided with the patch such as dosing instructions, child-resistant cartons, disposal pouches, titration cards, desiccants, temperature indicators, QR codes, multilingual instructions, or skin preparation wipes.

The term “in vitro dissolution” refers to laboratory testing of drug release from a patch matrix into buffer or simulated physiological solution over time, used to establish release characteristics and batch uniformity.

The term “Franz diffusion cell” refers to an apparatus for measuring permeation of drugs across skin or membrane samples, used for in vitro evaluation of transdermal patch delivery rates.

The term “batch-to-batch content uniformity” refers to consistency in drug content, weight, and thickness among patches produced in different manufacturing runs, assessed by representative sampling and analytical methods.

The term “score line” refers to a preformed physical feature on the release liner that facilitates easy and uniform removal of the liner from the adhesive side of the patch before application.

### Transdermal Drug Delivery Patch

The present invention provides for transdermal drug delivery systems designed to achieve controlled and sustained systemic delivery of tizanidine base through intact skin over extended time intervals. These patches include, in various embodiments, single-phase monolithic drug-in-adhesive matrices as well as alternative multilayer or reservoir constructions, offering flexibility for both formulation and drug release kinetics. Key innovations include the use of finely micronized or milled tizanidine base-free base or salt forms-uniformly dispersed within a pressure-sensitive adhesive matrix, optionally with one or more permeation enhancers, such as polyvinylpyrrolidone and diethylene glycol, to facilitate reliable skin absorption and consistent therapeutic plasma levels. The systems support precise content uniformity and minimal impurity levels, with a range of patch sizes and thicknesses suitable for titratable dosing across different patient populations. The patch may employ a combination of carefully selected adhesives, solvents, and barrier layers to ensure optimal skin adherence, pharmacokinetic performance, product stability, and robust protection from environmental exposure. Notably, the formulation and manufacturing methods overcome challenges of poor drug solubility, instability in certain solvents, and drug distribution uniformity, as demonstrated by rigorous in vitro, in vivo, and stability testing. The patches deliver therapeutically relevant amounts of drug over defined wear periods, providing a flexible platform suitable for adapting to other therapeutic actives or treatment regimens when desired.

### Monolithic Adhesive Matrix

The patches described herein may include a monolithic adhesive matrix, which consists of a continuous and homogeneous layer containing both the adhesive and the active pharmaceutical ingredient, with or without additional functional additives such as enhancers or solvents. This structure is formed by dispersing the drug and other excipients directly within a pressure-sensitive adhesive prior to application, avoiding any discrete reservoir or separate layered strata. The monolithic matrix may be fabricated from a variety of pressure-sensitive adhesives, including acrylic-based, silicone-based, or rubber-based chemistries, depending on compatibility with the active drug and desired release profile.

The matrix layer may incorporate the active drug in various forms—suspended, dissolved, or as micro-particulate—in order to optimize content uniformity and drug delivery efficiency. For specific embodiments, the use of acrylic adhesive (Duro-Tak 2051 or 2516) enables formation of a robust monolithic matrix with uniform dispersion of tizanidine base, yielding consistent mechanical properties and reproducible therapeutic performance. The construction facilitates straightforward, scalable manufacturing and may be adjusted in thickness, drug load, or additive content to meet differing clinical or regulatory specifications.

### Reservoir or Multilayer Systems

A transdermal drug delivery patch may alternatively feature a reservoir or multilayer construction, in which the device is composed of two or more spatially discrete domains, regions, or layers, each designed to serve a specific functional or pharmacokinetic role. At least one region may act as a reservoir containing a liquid, gel, semi-solid, or solid drug formulation, physically separated from other patch components such as the adhesive, release liner, or backing film. These designs enable temporal or spatial separation of formulation components-including drug, permeation enhancers, solvents, or additional excipients-thereby permitting finely tuned control over release kinetics, stability, and dual- or multi-phasic drug delivery profiles.

The reservoir may be realized as a pocket, cavity, or compartment formed within or between adhesive layers, or it may be encapsulated by a polymer membrane, film, or rate-controlling barrier made from materials such as ethylene-vinyl acetate (EVA), polyurethane, polyester, polyethylene, or other pharmaceutically acceptable polymers. Such barriers can be selected or engineered to modulate drug flux and release rates according to clinical requirements.

Multilayer systems may take forms including, but not limited to: (1) a drug-containing reservoir layer positioned adjacent to a rate-control membrane (e.g., EVA, polyurethane, or similar film) and overlayed with a separate peripheral or contact adhesive; (2) sequential or stratified layers of adhesives differing in composition, such as a drug-in-adhesive layer paired with an enhancer-in-adhesive or buffering layer; (3) dedicated sub-layers or interlayers for modulating moisture, oxygen, or enhancer permeation; and (4) combinations of solvent-based and solvent-free strata designed to provide staged, delayed, or biphasic drug release.

The reservoir or multilayer region may incorporate actives such as tizanidine base (free base or salt forms), with optional permeation enhancers (e.g., polyvinylpyrrolidone, diethylene glycol, fatty acids, plant oils, or other agents), as well as various matrix, gel, or polymeric compositions sufficient to support sustained, pulsed, or customizable release over defined durations ranging from 8 to 96 hours or more. Patch area may range from 1 cm² to 100 cm², supporting both individualized titration and broad clinical applications. Adhesive chemistries for various layers may be selected from acrylic, silicone, rubber, or any combination thereof, with or without additional functional additives, to optimize physical integrity, biocompatibility, and therapeutic performance based on the requirements of the target drug and use case.

### Pressure-Sensitive Adhesive

Pressure-sensitive adhesives (PSAs) suitable for the patch include a broad range of adhesive chemistries designed to provide tack and skin adherence at room temperature, without the need for activation by heat, moisture, or solvent. PSAs may be selected from acrylic copolymers, silicone-based adhesives, rubber-based adhesives, or other medically-acceptable adhesive systems.

Acrylic copolymer PSAs typically utilize polymer backbones derived predominantly from acrylic monomers, such as butyl acrylate, 2-ethylhexyl acrylate, methyl acrylate, or hydroxyethyl acrylate. These adhesives may further include copolymers bearing carboxyl (—COOH), hydroxyl (—OH), or other functional groups to modulate hydrophilicity, adhesion, and compatibility with drugs and excipients. Commercially available examples include, but are not limited to, Henkel Duro-Tak 2051 (carboxyl-functionalized) and Duro-Tak 2516 (hydroxyl-functionalized), which may be used alone or in blends, typically including 60-65% of total solids in the final adhesive matrix.

Alternatively, the PSA may be selected from non-acrylic chemistries, including silicone-based systems (e.g., polysiloxanes, silicone adhesives), rubber-based systems (e.g., polyisobutylene, styrene block copolymers), or other polymers compatible with pharmaceutical use. The use of non-acrylic PSAs may offer different drug release profiles, skin tolerability, or compatibility with specific permeation enhancers and active pharmaceutical ingredients.

The PSA component may also include heat-activated or moisture-activated adhesives if desired for specialized applications. Selection of the adhesive may be further tailored to avoid overlap with described acrylic copolymer systems, such as using unique polymer backbones, functional groups not disclosed herein, or novel manufacturing methods and crosslinking strategies, provided the adhesive delivers the requisite tack, flexibility, and compatibility with other patch components.

### Tizanidine Base

The active ingredient includes tizanidine base, provided in free base form and optionally processed into finely ground, micronized, or milled particulate matter to enhance dispersibility and dissolution. Particle size reduction may be achieved by mechanical milling, jet milling, or supplier-provided micronization, resulting in a median particle diameter (D90) less than 30 microns by volume, as measured by laser diffraction. Larger or unground particles may be included in broader variants to permit slower dissolution or release, but smaller particle size is preferred for enhanced skin permeation and content uniformity.

Embodiments may use tizanidine base derived from various synthetic routes, optionally further purified to reduce impurities, and may support batch-to-batch consistency by specification of supplier micronization and particle size testing prior to final product formulation.

Reference to the tizanidine base as having a specified particle size means that the tizanidine base employed in the preparation of the adhesive matrix had that particle size. The tizanidine base is typically introduced into the formulation as a micronized or particulate solid, which is suspended and/or partially dissolved in the solvent system during preparation of the adhesive matrix. Depending on solubility and process conditions, some fraction of tizanidine base may be present in dissolved molecular form in the matrix, while undissolved tizanidine base remains as particulate matter with the specified particle size distribution. Reference to particle size thus pertains to the undissolved tizanidine present in the final matrix. In some embodiments, all or substantially all of the tizanidine base remains as a suspended particulate phase, while in others, a combination of dissolved and suspended fractions may coexist, as dictated by formulation and processing variables.

### Permeation Enhancer

The adhesive matrix may include at least one permeation enhancer, selected from a broad range of chemical classes capable of increasing the flux of tizanidine or other active pharmaceutical ingredients across the skin. Suitable permeation enhancers encompass both hydrophilic and lipophilic agents, allowing flexibility in optimizing for different drugs, adhesives, and therapeutic needs. Examples include, but are not limited to, polyvinylpyrrolidone (povidone, various molecular weights such as K29/32 or K90), diethylene glycol (DEG), propylene glycol, polyethylene glycol, N-methyl pyrrolidone, fatty acids (e.g., oleic acid, caproic acid), essential oils (e.g., flaxseed oil, lemongrass oil, coriander oil), terpenes, citral, and other pharmaceutically acceptable excipients.

Enhancers may be included individually or in synergistic combinations, typically at 3-10% by weight of total matrix solids, with ratios and ingredient selection optimized for the active drug, targeted release profile, and matrix compatibility. The incorporation of both hydrophilic polymers (such as K29/32 povidone) and glycols (such as DEG) has been shown to provide enhanced permeation, stability, and content uniformity; however, alternative systems employing fatty acid derivatives, plant oils, or novel permeation strategies (such as ion-pairing or complexation with skin-penetrant cofactors) may also be used.

The selection, concentration, and combination of permeation enhancers can be tailored to achieve desired delivery characteristics, address potential irritation concerns, or to serve as a means of differentiation from previously described systems, further broadening the performance and intellectual property space available for transdermal patch development.

### Solvent System

Patch manufacture may utilize a solvent system including, e.g., ethanol and/or ethyl acetate to dissolve or disperse the drug, excipients, and adhesive during production. The ratio and selection of solvents may be adjusted to balance drug solubilization and adhesive compatibility-ethanol offering rapid evaporation and low toxicity and ethyl acetate providing moderate solvation and compatibility with various adhesives. The total content of solvent is generally sufficient to form a uniform coating solution and is substantially removed during drying, with a preferred residual solvent content below 500 ppm in the finished product.

Alternative volatile solvents may be evaluated, provided that they do not compromise product stability or regulatory compliance. The choice of ethanol and/or ethyl acetate is supported by their classification as USP/ICH Class 3 solvents, minimizing potential toxicological risk, and regulatory restrictions.

### Adhesive Matrix Thickness

The adhesive matrix may be applied and dried to form a layer of controlled thickness, generally about 0.013 inches, with a permissible range of variation ±0.004 inches. This thickness may be adjusted to accommodate differing drug loads, delivery duration, and patch sizes, while maintaining sufficient mechanical strength and skin adhesion. Measurement and adjustment of thickness is typically accomplished using precision micrometers at multiple locations across each patch or roll.

Specific manufacturing controls may include real-time monitoring and feedback adjustment to ensure that matrix thickness does not deviate more than ±10% from target set points, thereby supporting consistent drug loading, content uniformity, and delivery kinetics.

### Release Liner

A removable release liner may be composed of, e.g., polyester film, optionally surface-treated with a fluoropolymer layer to provide low surface energy and facilitate clean detachment from the adhesive matrix at the time of application. Suitable release liners include those having a thickness or caliper of approximately 2.9±0.02 mils, selected for compatibility with manufacturing processes, storage stability, and integrity during handling.

Compositional alternatives may include polyethylene terephthalate (PET) bases with various fluoropolymer coatings (e.g., polytetrafluoroethylene, fluorinated ethylene propylene), optionally die-cut or scored to promote user access. Commercially available options include 3M ScotchPak 9744 and 1022, both of which have demonstrated physical and chemical compatibility with patch adhesives and drugs of interest.

Alternatively, the removable release liner may be composed of, e.g., non-polyester film, optionally surface-treated with a non-fluoropolymers.

### Backing Film

The patch may be configured with a backing film including at least one polymeric layer, often a polyester or polyethylene film, optionally laminated with additional barrier layers such as metal (aluminum) vapor-deposited films or copolymers (e.g., EVA). The backing serves to physically support the adhesive matrix, provide occlusion (limit water vapor permeability), and protect the matrix from external environmental effects.

In specific embodiments, suitable backing films include multi-layered structures such as 3M ScotchPak 9738 (aluminized, occlusive) or 1012 (clear, flexible), with total thickness and flexibility tailored for comfort and cosmetic acceptability. The inclusion of a metallic barrier layer (e.g., aluminum vapor barrier) improves moisture resistance and drug stability during storage and wear.

Alternatively, the backing film is composed of all-metal or non-polymer backings.

### Patch Area

The patch may be manufactured in a range of discrete or continuous sizes, with the total surface area per unit selected to fall between 5 cm² and 20 cm². Variation in patch size facilitates titratable or individualized dosing, enabling therapeutic customization for patient needs or response. The boundary values (5-20 cm²) may be expanded or subdivided for pediatric or special populations.

Multiple patch sizes may be included in a commercial kit or product line, supporting incremental upward or downward titration of drug dose while preserving uniformity of other patch characteristics (thickness, coat weight, etc.) across sizes.

### Acrylic Copolymer Functional Groups (in Adhesive)

The pressure-sensitive adhesive layer may be included of an acrylic copolymer bearing either carboxyl (—COOH) or hydroxyl (—OH) functional groups. The presence of these chemical moieties influences adhesive characteristics such as hydrophilicity, hydrogen bonding, and compatibility with functional excipients and drugs. These adhesives may be derived from the copolymerization of acrylic acid, hydroxyethyl acrylate, or related monomers with other acrylic co-monomers.

Specific illustrative adhesives include Duro-Tak 2051 (carboxyl functionalized) and Duro-Tak 2516 (hydroxyl functionalized), chosen on their ability to enable consistent active drug incorporation, stable matrix formation, and enhanced permeation when used in conjunction with penetration enhancers.

### Tizanidine Particle Size

Tizanidine base may be processed such that at least 90% of particles present (by volume) have a diameter less than 30 microns, as measured by suitable particle sizing methodologies (e.g., laser diffraction). Micronization improves content uniformity and dissolution kinetics within the transdermal matrix, contributing to reproducible delivery and small patch size options.

Supplier-controlled micronization provides for uniform batch quality and enables robust scale-up operations. The system may further allow for quality control testing of finished lots to ensure continued compliance with the specified particle size distribution for effective permeation and content uniformity.

### Content Uniformity

The patch may be fabricated such that the distribution of tizanidine base throughout the matrix is uniform to within ±5% of the mean content, both across the surface of a single patch and among patches within a batch. This uniformity is achieved via homogenization, controlled mixing, and defined order of addition of components during manufacture, with in-process analytical confirmation (e.g., via HPLC assay of extracted samples or mass uniformity testing).

Content uniformity ensures dose accuracy, minimizes clinical variability, and supports regulatory compliance. The content specification may be broadened (e.g., ±10%) for investigational or early-stage products where necessary, but tight uniformity increases batch-to-batch reliability.

### Patch Size

The patch system may be commercialized as a set of multiple discrete sizes, each with defined surface area in the range of 5 cm² to 20 cm², to provide user-selectable, titratable dosing. Patches of differing sizes may each deliver a proportional amount of the active pharmaceutical ingredient, with identical drug concentration per unit area and matrix composition across the size range.

Dosing regimens may include protocols wherein patients begin therapy at the lowest size and titrate upward as needed, with patch replacement occurring at defined intervals to tailor therapy. This approach simplifies personalized medicine and accommodates a broad spectrum of patient characteristics or clinical needs.

### Drug Load in Adhesive Matrix

Formulations may include tizanidine base at a concentration ranging from about 8% to about 16% (w/w) of the total solids content of the adhesive matrix, exclusive of volatile solvents lost during drying. Drug load optimization addresses solubility, stability, skin permeation, and manufacturability. Selected embodiments use loads of 12-15% for optimal batch-to-batch consistency and delivery performance.

Uniformity of drug load within each batch is maintained by rigorous process controls, validated weighing and mixing protocols, and in-process analytical verification, ensuring the loaded content fits within ±5% of specification per patch.

### Adhesive Matrix Thickness

The adhesive matrix can be manufactured to a nominal thickness of, e.g., 0.013 inches, with allowed variance of 10%, thereby targeting uniform drug and enhancer content across the patch and achieving desired mechanical and delivery properties. Thickness can be monitored via digital micrometer, and production lines may include in-process thickness measurement at multiple points to ensure batch uniformity.

Thicker or thinner matrices may be produced for special applications (e.g., rapid-release, pediatric dosing), but the described target provides a balance between skin adhesion, comfort, and sustained delivery for the intended therapeutic use.

### Impurity Levels

The patch formulation and packaging may be configured such that total process-related and degradation impurities (as measured by validated HPLC protocols) do not exceed, e.g., 0.1% by weight, even after storage under ambient or light-protected conditions for a minimum duration of 12 months. This is achieved through the selection of compatible solvents and adhesives, exclusion of impurity-promoting materials (e.g., acetone), and use of robust, occlusive packaging materials.

Extended stability data, including periodic impurity testing at regular intervals (monthly, quarterly), provide assurance of product integrity and safety throughout labeled shelf life, supporting regulatory acceptance and clinical use.

### Coat Weight Per Patch Area

Patches can be produced with a defined coat weight, e.g., 0.045 grams (±10%) of adhesive matrix per 5 cm² of patch area, exclusive of the release liner and backing film. The coat weight can be confirmed by mass measurement of cut samples, subtracting component weights, and supports accurate drug content and delivery profile.

Alternative weights and corresponding drug loads may be implemented for different patch sizes or dose requirements, provided that supporting physical and pharmacokinetic requirements are met.

### Enhancer

Formulations may employ, e.g., both polyvinylpyrrolidone (PVP, povidone) and diethylene glycol (DEG) as penetration enhancers within the adhesive matrix, at concentrations and ratios optimized to maximize tizanidine flux across human skin. PVP may be provided in molecular weight grades K29/32 or K90, at levels of 3-5% of total solids, and DEG at 3-9%, based on deliverability studies.

The co-use of these enhancers offers synergistic permeation benefits and reproducible delivery profiles, while remaining compatible with processing and stability requirements across a range of patch designs.

### Residual Solvent Content

The final dry adhesive matrix is specified to retain, e.g., less than 500 ppm of certain solvents classified as process or residual contaminants (e.g., acetone, chloroform, ethyl acetate, ethanol), as determined by gas chromatography. The low-residual content can be achieved through controlled drying and validated analytical release criteria.

Broader solvent content specifications may be included to satisfy global regulatory requirements. In all cases, batch testing confirms that residual solvents do not exceed limits established by ICH, USP, or other relevant standards.

### Release Liner

The removable release liner may be formed, e.g., as a polyester (e.g., PET) film, coated with a fluoropolymer, and produced with a caliper (thickness) of approximately 2.9 mils (±0.02 mils). The selection of liner thickness supports ease of handling during manufacturing and end-user application, while ensuring adequate stiffness for automated die-cutting and packaging processes.

Broader embodiments may include liners of different thickness, materials, or coatings, so long as the function of clean removal and compatibility with the adhesive are maintained.

### Backing Film

The patch may include a multi-layer backing film construction composed of at least one polymeric base layer (e.g., polyester, polyethylene) in combination with a metallic vapor-deposited barrier layer, typically aluminum. The inclusion of the metalized layer restricts water vapor and oxygen transmission, serves to enhance patch stability, and prevents loss of volatiles or ingress of environmental contaminants.

Manufacturing flexibility is provided by sourcing pre-laminated films or by co-extrusion processes, permitting variations in thickness, color, or occlusivity as dictated by product requirements.

### Patch Geometry

Patches may be die-cut or otherwise formed with a periphery approximating a rectangle, whose corners are smoothly rounded rather than sharply defined. This configuration enhances user comfort, reduces edge lift during wear, and minimizes accidental removal.

Rectangular-with-rounded-corner geometry additionally supports efficient nesting and material utilization during die-cutting, enabling cost-effective mass production and packaging. Other geometric variants (e.g., oval, circular) may be employed depending on use case preference.

### Variation in Tizanidine Content

The process is controlled such that the variation in tizanidine base content at any point within a single patch does not exceed, e.g., ±5% of the mean for that patch. Uniform mixing, precision spraying or spreading, and continuous in-process sampling facilitate this stringent specification.

Larger or smaller tolerances may be introduced for early-stage development, but product intended for commercial distribution is preferably held to the ±5% criterion, with compliance ensured by validated QC protocols.

### Total Related Substance Impurities

The patch matrix and all its components are specified such that related substance impurities, measured by HPLC and according to regulatory guidelines (e.g., USP, ICH Q3A), remain below, e.g., 0.1% by weight in the finished product. This limit applies both at release and at the end of the labeled shelf life.

Analytical methodologies are validated for sensitivity and specificity to known impurities of tizanidine, and process steps are optimized to avoid impurity formation, particularly by avoiding reactive solvents or excipient incompatibilities.

### Release Liner Score Line for Removal

The release liner may be provided with a physical indentation, score, or printed line across its surface to facilitate partial or full separation and easy removal by the end-user prior to patch application. The score may extend fully or partially across the liner, positioned to aid peel initiation without risk of damage to the adhesive matrix.

Versions may include single, double, or finger-tabbed score lines, as desired for optimal usability across target populations. The production method enables repeatable and controlled scoring concurrent with die-cutting operations.

### Occlusive Backing

The backing film may be specified to provide an occlusive barrier to moisture vapor transmission, quantified by an MVTR (moisture vapor transmission rate) less than, e.g., 1 g/m²/24 hours under standardized test conditions. The occlusivity minimizes water loss from the skin, protects the drug matrix from environmental humidity, and contributes to patch stability.

The inclusion of aluminum vapor barriers or multi-layer construction may be used to achieve and document target MVTR performance, supporting global use and distribution in varied climates.

### In-Process Micrometer Measurement of Coat Thickness

Production protocols include continuous or sampling-based micrometer measurements of matrix thickness throughout coating, drying, and laminating steps, ensuring coat thickness deviation does not exceed, e.g., ±10% of the target value. Measurement at multiple positions (center, left, right; start, middle, end of roll) confirms both within-patch and batch-to-batch uniformity.

Automated or semi-automated thickness monitoring may further be employed to flag deviations and trigger corrective actions during production runs.

### Immediate Lamination to Backing Film After Drying

The process includes a stepwise sequence wherein the adhesive drug matrix, once dried, is immediately laminated to the backing film with no intermediate storage or holding period. This “wet-to-dry” transfer prevents variability in adhesion or matrix consistency that may arise from moisture uptake or incomplete drying.

Continuous inline lamination maximizes process efficiency and minimizes sources of variability, contributing to overall uniformity and product stability.

### Patch Stability

Long-term and accelerated stability protocols demonstrate that the patch, when stored shielded from light (e.g., in its primary pouch) at ambient temperature for at least, e.g., 12 months, maintains total related substance impurity levels below, e.g., 0.1% by validated HPLC. Physical integrity and delivery performance are likewise unchanged by storage conditions.

Extended stability data support product registration in diverse markets and provide a scientific basis for labeled shelf life and recommended storage conditions.

### Patch Size

Manufacturing may include creation of multiple patch sizes, such as, e.g., 5 cm², 10 cm², and 20 cm², as discrete, individually packaged products, to allow clinicians and patients to titrate the dose upward or downward without need for cutting or modification. All sizes share the same composition and matrix structure, differing only in geometric area and proportional drug quantity delivered.

Provision of a range of sizes accommodates interpatient differences in body weight, symptom severity, and metabolic needs, and may support pediatric, adult, and geriatric populations alike.

### Selection of Release Liner

The release liner for the patch may be optionally selected from commercially available fluoropolymer-coated polyester films, specifically including, e.g., 3M ScotchPak 9744 or 1022, each of which is compatible with the described adhesive formulations and enables efficient release and transfer of the patch to the skin.

Selection may be based on factors such as availability, cost, or liner surface energy, with the option to qualify new materials as needed for global sourcing flexibility.

### Sequential Mixing

The adhesive matrix may be prepared by a specified sequence of addition, wherein, e.g., ethanol is added first, followed in sequence by povidone, diethylene glycol, and tizanidine base, with thorough mixing at each step, and finally by slow incorporation of the adhesive. This order supports optimal dispersibility and prevents agglomeration or loss of uniformity.

Adherence to a controlled order of addition is empirically supported for maximizing patch performance, reproducibility, and minimizing batch-to-batch variability.

### Tizanidine Base Dispersed as Particulate Suspension Prior to Coating and Drying

The active tizanidine base can be included in the coating solution as a finely dispersed suspension in the selected solvents and additives, prior to the addition of adhesive and subsequent coating/drying. This method assures that the drug remains evenly distributed, is not dissolved or solubilized to excess, and facilitates control over release kinetics and particle size attributes.

Particulate dispersion may employ high-shear mixing or homogenization and is monitored for particle size and distribution prior to coating.

Method of Treating Increased Muscle Tone Associated with Spasticity

Disclosed herein are methods for reducing muscle spasticity and associated symptoms by transdermally administering tizanidine base using the provided patch systems. The methods include selection and application of patches to achieve sustained release and steady-state plasma concentrations within the therapeutic window, thereby avoiding the pronounced fluctuations-such as peak-related side effects or fed/unfed variability-often observed with oral formulations.

Treatment protocols are designed to accommodate individualized titration and dosing intervals, with the option for initial, maintenance, and adjustment phases as needed for effective management of muscle tone. Importantly, these approaches are suitable for patient groups who are unable or unwilling to swallow, are intolerant of oral therapy, or are at increased risk of gastrointestinal adverse effects.

The method achieves consistent drug exposure, circumvents hepatic first-pass metabolism, and enables therapy in diverse clinical situations, independent of food intake or circadian timing. Unexpectedly, application of these transdermal systems results in a reduced incidence of adverse gastrointestinal reactions and a lower Cmax while maintaining area under the curve (AUC) bioequivalence to established oral dosing. The flexibility in dose titration and wear intervals further allow these methods to be personalized to the needs of distinct patient populations.

### Release Properties

Patch composition and structure may be optimized to release tizanidine base at a sustained and controlled rate, delivering an effective drug dose transdermally over a minimum period of 24 to 48 hours. Sustained-release kinetics may be achieved via selection of adhesive chemistry, drug particle size, permeation enhancers, and matrix thickness, alone or in combination. The system may include pharmacokinetic properties that match or approximate the area under the concentration-time curve (AUC) of conventional oral administration, but with a steadier, prolonged delivery and reduced Cmax.

Control of release may extend to longer durations (e.g., 72 hours), by adjusting formulation parameters described herein. The system is configured such that removal and reapplication are straightforward for end-users at the conclusion of each dosing period.

### Patch Delivery Profile

Formulations are developed to deliver, e.g., approximately 3 mg (±10%) of tizanidine base over a 48-hour wear period, with delivery profiles demonstrating linear cumulative release as measured in vitro and in vivo. Pharmacokinetic studies confirm area under the curve (AUC) comparable to an equivalent oral dose, but with lower Cmax and fewer gastrointestinal side effects. Further, the transdermal system is unaffected by the patient's fed or unfed state, providing consistent systemic exposure.

Release profiles and AUC matching are validated by analytical and pharmacokinetic means, and clinical data support the reduction or elimination of dose-related spikes and side effects associated with oral administration.

### Pharmacokinetics and Clinical Advantages

The designed patch system, when applied as directed, can deliver tizanidine base transdermally at a rate establishing steady-state plasma concentrations characterized by lower Cmax than equivalent oral dosing. Further, the systemic exposure exhibits minimal variability due to oral feeding state, and clinical studies confirm reduced incidence of gastrointestinal side effects relative to oral formulations.

These pharmacokinetic and clinical advantages are attributable to controlled-release matrix composition, bypass of hepatic first-pass metabolism, and enhancer-mediated skin permeation. In-use and in vitro data confirm reproducibility of such effects regardless of patient or environmental state at the time of application.

### Application Method

Therapeutic application may be achieved by adhering the patch to an appropriate skin site and maintaining contact for an interval of, e.g., at least 48 hours, during which time sustained delivery is achieved and product performance is maintained. Wear periods of 24, 72, or more hours are enabled by adjusting matrix thickness, composition, and backing film.

Instructions for Use specify steps necessary for application, removal, handling, and monitoring throughout the prescribed duration.

### Method Reducing or Eliminating GI Side Effects Versus Oral Tizanidine

Administration of tizanidine base via the described transdermal patch, as opposed to oral tablets or capsules, can substantially reduce or eliminates gastrointestinal side effects, such as abdominal pain or diarrhea, by avoiding direct exposure of the gut mucosa and first-pass hepatic metabolism.

Such improved safety may be claimed for all patient populations, particularly those previously intolerant to oral regimens.

### Treating Patients Intolerant or Unable to Take Oral Medication

The described method and patch system can be indicated for use in patients with difficulty swallowing (dysphagia), intolerance to oral medication, or contraindications to GI absorption, such as severe nausea, postoperative status, or feeding tube placement. Application to intact skin enables effective therapy without reliance on oral or enteral routes.

Professional guidelines may recommend patch therapy as first-line for these populations, and package labeling or kit instructions may address these indications.

### Method Avoiding Pharmacokinetic Fed/Unfed Variability

By delivering tizanidine base transdermally, the patch can avoid pharmacokinetic fluctuations associated with fed vs. fasted state at the time of dosing. Transdermal absorption is unaffected by meals, ensuring consistent and predictable exposure for clinical effect.

Comparative studies using fasted/fed crossovers confirm that patch use results in negligible variability due to food intake, supporting expanded indications and patient reassurance.

### Time to Therapeutic Effect

The system can be tailored such that onset of therapeutic effect, e.g., reduction of muscle spasticity, commences between, e.g., 2 and 8 hours following patch application, as determined by pharmacokinetic and pharmacodynamic studies. Formulation factors (drug load, matrix, enhancer types/levels) may be modulated to tune the onset profile for various clinical needs.

Extended or delayed onset versions may also be supported, for overnight, circadian, or chronic control indications.

### Dosing Protocol

Recommended protocols include applying patches at times aligned to individual circadian rhythm or symptoms (e.g., nighttime spasticity relief) and adjusting dose upward or downward by increments (e.g., 5 cm² patches added or removed) as needed. The patch may be worn for intervals ranging from 24 to 96 hours per application, with maximum cumulative daily dose generally not exceeding 36 mg/24 hr equivalent (summed across all concurrent patches in use).

Instructions provide detailed guidance for titration, adjustment, and safe maximum durations and quantities.

### Bypassing Hepatic First-Pass Metabolism

Transdermal delivery by the patch can avoid the hepatic first-pass effect characteristic of oral administration, thereby increasing systemic bioavailability and permitting therapeutic effect at a lower dose relative to oral regimens. Dose reduction reduces risk of side effects, toxicity, or drug-drug interactions related to hepatic metabolism.

Mechanistically, the system thus facilitates novel therapy for patients with impaired metabolism, polypharmacy, or age-related changes in pharmacokinetics.

### Commercial Kit

The invention further provides flexible and comprehensive kits for clinical or home use, facilitating safe and effective administration of tizanidine base through the provided transdermal patches. Each kit is configured to contain one or more individually packaged patches, sealed within protective, light-impermeable pouches to ensure product stability and longevity. Kits are supplemented with clear written and visual instructions for use, and may further include additional components such as child-resistant packaging, titration guides, dosing schedules, skin preparation wipes, disposal pouches, desiccants, temperature indicators, and digital resources to optimize patient adherence, storage, and disposal.

The kit design accommodates multi-language, home, hospital, or pharmacy dispensing, and provides a modular framework that readily integrates future clinical or regulatory modifications. By combining delivery devices with comprehensive support materials, the described kits improve user experience, minimize medication errors, enhance safety, and support customized titration protocols for a range of patient requirements.

A commercial kit includes at least one individually packaged patch, sealed in a light-protective pouch, together with written and/or visual instructions for proper application, handling, and disposal of the patch. The kit may be packaged in a carton designed for at-home, hospital, or pharmacy dispensing, and may be provided in single-use, multi-use, or trial-size configurations.

Instructions are prepared for lay or medical user comprehension, with diagrams or QR codes linking to instructional videos or digital resources.

The kit may further include one or more supplemental components, including a child-resistant outer carton, calendar or dosing schedule, separate disposal pouch for used patches, titration guide or dosing card, desiccant (to control humidity), temperature indicator (for storage monitoring), QR code or link for digital instructions and adherence aids, multi-language printed materials, and sterile wipes or pads for skin preparation and infection control.

Configurations are varied for clinical, home, or research needs, and contents may be customized at packaging, prescription, or distribution.

### Patch Packaging

Each individual patch may be enclosed in a sealed pouch composed of light-impermeable materials (e.g., aluminum laminate, opaque polymer), protecting product integrity against photodegradation throughout storage and transport. The pouch design may further be heat-sealed on all sides to exclude moisture and oxygen.

Alternative pouch configurations—including those with printed UM/barcodes, instructions, or temperature indicators—may be used, provided light protection remains.

### Written and Visual Instructions

Comprehensive instructions, provided in both written and visual form, guide the end-user through patch application and removal, titration of dose, and safe disposal. Visual instructions may include diagrams, photographs, or infographics demonstrating each step, with translated materials available for global distribution.

Instructions may also be embedded in or linked via QR code, enabling continuous updates, multi-platform access, and improved user adherence and safety.

### Specific Ranges, Values, and Embodiments

The specific embodiments describing the ranges and values provided below are for illustration purposes only, and do not otherwise limit the scope of the disclosed subject matter, as defined by the claims.

In specific embodiments, the transdermal drug delivery patch includes (1) an adhesive matrix containing a pressure-sensitive adhesive; (2) tizanidine base as the active pharmaceutical ingredient, the tizanidine base dispersed within the adhesive matrix; (3) at least one permeation enhancer; and (4) solvent system.

In specific embodiments, the adhesive matrix is monolithic.

In specific embodiments, the adhesive matrix has a thickness of 0.013±0.005 inches.

In specific embodiments, the adhesive matrix has a thickness of 0.013±0.004 inches.

In specific embodiments, the adhesive matrix has a thickness of 0.013±0.003 inches.

In specific embodiments, the adhesive matrix has a thickness of 0.013±0.002 inches.

In specific embodiments, the adhesive matrix has a thickness of 0.013±0.001 inches.

In specific embodiments, the adhesive matrix has a thickness of 0.013 inches.

In specific embodiments, the adhesive matrix layer has a thickness of about 0.013 inches, ±12.5%.

In specific embodiments, the adhesive matrix layer has a thickness of about 0.013 inches, ±10%.

In specific embodiments, the adhesive matrix layer has a thickness of about 0.013 inches, ±7.5%.

In specific embodiments, the adhesive matrix layer has a thickness of about 0.013 inches, ±5%.

In specific embodiments, the adhesive matrix layer contains <750 ppm acetone as residual solvent.

In specific embodiments, the adhesive matrix layer contains <500 ppm acetone as residual solvent.

In specific embodiments, the adhesive matrix layer contains <250 ppm acetone as residual solvent.

In specific embodiments, the adhesive matrix layer contains <750 ppm chloroform as residual solvent.

In specific embodiments, the adhesive matrix layer contains <500 ppm chloroform as residual solvent.

In specific embodiments, the adhesive matrix layer contains <250 ppm chloroform as residual solvent.

In specific embodiments, the adhesive matrix layer contains <500 ppm acetone and <500 ppm chloroform as residual solvents.

In specific embodiments, the total related substance impurities in the adhesive matrix layer are <0.25% by weight as measured by high-performance liquid chromatography.

In specific embodiments, the total related substance impurities in the adhesive matrix layer are <0.1% by weight as measured by high-performance liquid chromatography.

In specific embodiments, the total related substance impurities in the adhesive matrix layer are <0.075% by weight as measured by high-performance liquid chromatography.

In specific embodiments, the total related substance impurities in the adhesive matrix layer are <0.05% by weight as measured by high-performance liquid chromatography.

In specific embodiments, the adhesive matrix is produced by a process that includes in-process micrometer measurement of coating thickness.

In specific embodiments, the adhesive matrix has a coat thickness variation not exceeding ±1²0.5% across the patch.

In specific embodiments, the adhesive matrix has a coat thickness variation not exceeding ±10% across the patch.

In specific embodiments, the adhesive matrix has a coat thickness variation not exceeding ±7.5% across the patch.

In specific embodiments, the adhesive matrix is produced by a process that includes sequential mixing of ingredients and immediate lamination to the backing film following drying, with no intermediate hold period.

In specific embodiments, preparation of the adhesive matrix includes the sequential addition of ethanol, povidone, diethylene glycol, tizanidine base, and, finally, adhesive.

In specific embodiments, the adhesive includes an acrylic pressure-sensitive adhesive.

In specific embodiments, the adhesive includes an acrylic copolymer.

In specific embodiments, the adhesive includes an acrylic copolymer having a —COOH or —OH functional group.

In specific embodiments, the adhesive includes an acrylic copolymer having at least one of a —COH and —OH functional groups.

In specific embodiments, the adhesive includes an acrylic copolymer having —COOH and —OH functional groups.

In specific embodiments, the tizanidine base is finely ground, micronized, or milled.

In specific embodiments, the tizanidine base present in the adhesive matrix is micronized to a particle size <50 microns D90 (by volume) as measured by laser diffraction.

In specific embodiments, the tizanidine base present in the adhesive matrix is micronized to a particle size <40 microns D90 (by volume) as measured by laser diffraction.

In specific embodiments, the tizanidine base present in the adhesive matrix is micronized to a particle size <30 microns D90 (by volume) as measured by laser diffraction.

In specific embodiments, the tizanidine base present in the adhesive matrix is micronized to a particle size <25 microns D90 (by volume) as measured by laser diffraction.

In specific embodiments, the tizanidine base present in the adhesive matrix is micronized to a particle size <20 microns D90 (by volume) as measured by laser diffraction.

In specific embodiments, the tizanidine base employed in the preparation of the adhesive matrix are friable crystals.

In specific embodiments, the tizanidine base employed in the preparation of the adhesive matrix is micronized.

In specific embodiments, the drug load in the adhesive matrix includes between about 8% and about 16% by weight of the solids in the adhesive layer.

In specific embodiments, the drug load in the adhesive matrix includes between about 9% and about 15% by weight of the solids in the adhesive layer.

In specific embodiments, the drug load in the adhesive matrix includes between about 10% and about 15% by weight of the solids in the adhesive layer.

In specific embodiments, the drug load in the adhesive matrix includes 12±6 wt. % of the solids in the adhesive layer.

In specific embodiments, the drug load in the adhesive matrix includes 12±5 wt. % of the solids in the adhesive layer.

In specific embodiments, the drug load in the adhesive matrix includes 12±4 wt. % of the solids in the adhesive layer.

In specific embodiments, the drug load in the adhesive matrix includes 12±3 wt. % of the solids in the adhesive layer.

In specific embodiments, the drug load in the adhesive matrix includes 12±2 wt. % of the solids in the adhesive layer.

In specific embodiments, the drug content is uniform to within ±7.5% across individual patches and batches.

In specific embodiments, the drug content is uniform to within ±5% across individual patches and batches.

In specific embodiments, the drug content is uniform to within ±2.5% across individual patches and batches.

In specific embodiments, the tizanidine base content varies by no more than ±7.5% across the patch.

In specific embodiments, the tizanidine base content varies by no more than ±5% across the patch.

In specific embodiments, the tizanidine base content varies by no more than ±2.5% across the patch.

In specific embodiments, the tizanidine base content uniformity is maintained within ±7.5% across the patch and batch.

In specific embodiments, the tizanidine base content uniformity is maintained within ±5% across the patch and batch.

In specific embodiments, the tizanidine base content uniformity is maintained within ±2.5% across the patch and batch.

In specific embodiments, the tizanidine base content uniformity is maintained within ±1% across the patch and batch.

In specific embodiments, the at least one permeation enhancer includes at least one of povidone and diethylene glycol.

In specific embodiments, the at least one permeation enhancer includes povidone and diethylene glycol.

In specific embodiments, the at least one permeation enhancer includes at least one of polyvinylpyrrolidone and diethylene glycol.

In specific embodiments, the at least one permeation enhancer includes polyvinylpyrrolidone and diethylene glycol.

In specific embodiments, the solvent system was present during preparation of the adhesive matrix.

In specific embodiments, the solvent system was employed during preparation of the adhesive matrix.

In specific embodiments, the solvent system includes at least one of ethanol and ethyl acetate.

In specific embodiments, the solvent system includes ethanol and ethyl acetate.

In specific embodiments, the solvent system includes <500 ppm of ethyl acetate as residual solvent.

In specific embodiments, the solvent system includes <250 ppm of ethyl acetate as residual solvent.

In specific embodiments, the solvent system includes <500 ppm of ethanol as residual solvent.

In specific embodiments, the solvent system includes <250 ppm of ethanol as residual solvent.

In specific embodiments, the solvent system includes <500 ppm of ethyl acetate and <500 ppm of ethanol as residual solvents.

In specific embodiments, the solvent system includes <250 ppm of ethyl acetate and <250 ppm of ethanol as residual solvents.

In specific embodiments, a release liner is in contact with the adhesive matrix.

In specific embodiments, the release liner includes a fluoropolymer-coated polyester film.

In specific embodiments, the release liner includes a fluoropolymer-coated polyester film having a caliper of 2.9±0.04 mils.

In specific embodiments, the release liner includes a fluoropolymer-coated polyester film having a caliper of 2.9±0.03 mils.

In specific embodiments, the release liner includes a fluoropolymer-coated polyester film having a caliper of 2.9±0.02 mils.

In specific embodiments, the release liner includes a fluoropolymer-coated polyester film having a caliper of 2.9±0.01 mils.

In specific embodiments, the release liner is provided with a score line extending across the patch to facilitate removal of the liner from the adhesive matrix.

In specific embodiments, release liner includes at least one of 3M ScotchPak 9744 and ScotchPak 1022 fluoropolymer-coated polyester films.

In specific embodiments, release liner includes 3M ScotchPak 9744 or ScotchPak 1022 fluoropolymer-coated polyester films.

In specific embodiments, a backing film is attached to the opposite side of the patch from the release liner.

In specific embodiments, a backing film is attached to the opposite side of the patch from the release liner, providing support, occlusivity, and protection for the adhesive matrix.

In specific embodiments, the backing film includes a laminate containing at least one polymeric film and a barrier layer.

In specific embodiments, the backing film includes a multi-layer laminate.

In specific embodiments, the backing film includes a multi-layer laminate that includes at least one of a polymeric film and a metallic vapor barrier layer.

In specific embodiments, the backing film includes a multi-layer laminate that includes a polymeric film and a metallic vapor barrier layer.

In specific embodiments, the backing film is substantially occlusive.

In specific embodiments, the backing film is substantially occlusive, having a moisture vapor transmission rate (MVTR) of less than 2.5 g/m²/24 hr.

In specific embodiments, the backing film is substantially occlusive, having a moisture vapor transmission rate (MVTR) of less than 1 g/m²/24 hr.

In specific embodiments, the backing film is substantially occlusive, having a moisture vapor transmission rate (MVTR) of less than 0.75 g/m²/24 hr.

In specific embodiments, the patch has an area between 5 cm² and 20 cm².

In specific embodiments, the patch has an area of 12.5±7.5 cm².

In specific embodiments, the patch has an area of 12.5±7 cm².

In specific embodiments, the patch has an area of 12.5±5 cm².

In specific embodiments, the patch has an area of 12.5±3 cm².

In specific embodiments, the patch has an area of 12.5±2.5 cm².

In specific embodiments, the patch is provided in a plurality of titratable sizes between 5 cm² and about 20 cm².

In specific embodiments, the patch is provided in a plurality of titratable sizes of 12.5 ±7.5 cm².

In specific embodiments, the patch is provided in a plurality of titratable sizes of 12.5±7 cm².

In specific embodiments, the patch is provided in a plurality of titratable sizes of 12.5±5 cm².

In specific embodiments, the patch is provided in a plurality of titratable sizes of 12.5±3 cm².

In specific embodiments, the patch is provided in a plurality of titratable sizes of 12.5±2.5 cm².

In specific embodiments, the patch is adapted to release tizanidine base transdermally over a period of time.

In specific embodiments, the patch is adapted to release tizanidine base transdermally over a period of at least 24 hours.

In specific embodiments, the patch is adapted to release tizanidine base transdermally over a period of at least 48 hours.

In specific embodiments, the patch is adapted to release tizanidine base transdermally over a period of 24 to 48 hours.

In specific embodiments, the patch is adapted to release tizanidine base transdermally over a period of at least 24 to 48 hours.

In specific embodiments, the patch is configured such that tizanidine base content is uniform.

In specific embodiments, the patch is configured such that tizanidine base content is uniform to within ±7.5% across the patch.

In specific embodiments, the patch is configured such that tizanidine base content is uniform to within ±5% across the patch.

In specific embodiments, the patch is configured such that tizanidine base content is uniform to within ±2.5% across the patch.

In specific embodiments, the patch is manufactured and stored in a manner maintaining <0.25% total impurities as measured by HPLC for at least 12 months storage at ambient temperature and in darkness.

In specific embodiments, the patch is manufactured and stored in a manner maintaining <0.1% total impurities as measured by HPLC for at least 12 months storage at ambient temperature and in darkness.

In specific embodiments, the patch is manufactured and stored in a manner maintaining <0.075% total impurities as measured by HPLC for at least 12 months storage at ambient temperature and in darkness.

In specific embodiments, the patch is manufactured and stored in a manner maintaining <0.05% total impurities as measured by HPLC for at least 12 months storage at ambient temperature and in darkness.

In specific embodiments, wherein a 5 cm² sample of the patch has a coat weight of about 0.045 grams, ±12.5%.

In specific embodiments, wherein a 5 cm² sample of the patch has a coat weight of about 0.045 grams, ±10%.

In specific embodiments, wherein a 5 cm² sample of the patch has a coat weight of about 0.045 grams, ±7.5%.

In specific embodiments, the patch is rectangular in shape with rounded corners.

In specific embodiments, the patch delivers about 3 mg of tizanidine base, ±12.5%, over a period of 48 hours under neutral pH conditions.

In specific embodiments, the patch delivers about 3 mg of tizanidine base, ±10%, over a period of 48 hours under neutral pH conditions.

In specific embodiments, the patch delivers about 3 mg of tizanidine base, ±7.5%, over a period of 48 hours under neutral pH conditions.

In specific embodiments, the patch achieves an in vitro or in vivo dissolution profile with linear cumulative delivery over 24-48 hours.

In specific embodiments, the patch achieves a release profile matching or approximating the area under the concentration-time curve (AUC) of an equivalent oral dose without corresponding Cmax spikes or GI side effects.

In specific embodiments, the patch maintains consistent release kinetics regardless of feeding state.

In specific embodiments, the patch is packaged in a light-impermeable pouch to minimize photodegradation of the tizanidine base.

In specific embodiments, the patch remains stable with no increase in total impurities above 0.25% after storage at ambient temperature shielded from light for a period of at least 12 months.

In specific embodiments, the patch remains stable with no increase in total impurities above 0.1% after storage at ambient temperature shielded from light for a period of at least 12 months.

In specific embodiments, the patch remains stable with no increase in total impurities above 0.75% after storage at ambient temperature shielded from light for a period of at least 12 months.

In specific embodiments, the patch shows no loss in therapeutic performance over its labeled shelf life.

In specific embodiments, the patch shows ≤20% loss in therapeutic performance over its labeled shelf life.

In specific embodiments, the patch shows ≤10% loss in therapeutic performance over its labeled shelf life.

In specific embodiments, the patch shows ≤5% loss in therapeutic performance over its labeled shelf life.

In specific embodiments, the patch shows ≤2.5% loss in therapeutic performance over its labeled shelf life.

In specific embodiments, the patch shows ≤1% loss in therapeutic performance over its labeled shelf life.

In specific embodiments, the patch is produced in a plurality of discrete sizes between 5 cm² and 20 cm² to provide titratable dosing.

In specific embodiments, the patch is uniquely sized (e.g., <5 cm² for pediatric use, or >20 cm² for refractory/high-need patients, up to 100 cm²).

In specific embodiments, the patch is sized <5 cm² for pediatric use.

In specific embodiments, the patch is sized >20 cm² for refractory/high-need patients.

In specific embodiments, the patch is sized >20 cm² for refractory/high-need patients, up to 100 cm²).

In specific embodiments, the patch is sized up to 100 cm² for refractory/high-need patients.

In specific embodiments, during manufacture of the patch, the coating is laminated to the backing film immediately after drying without any hold time.

In specific embodiments, during manufacture of the patch, the tizanidine base is dispersed as a particulate suspension within the solvent-based adhesive formulation prior to coating and drying.

In specific embodiments, the patch, when used as intended, achieves a steady-state plasma tizanidine base level with a Cmax lower than that of an equivalent orally administered dose,

In specific embodiments, the patch, when used as intended, delivers tizanidine base with a reduced incidence of gastrointestinal side effects compared to oral administration, including in patients sensitive or intolerant to oral therapy, and/or in those exhibiting pharmacokinetic variability with oral administration.

In specific embodiments, the patch, when used as intended, treats increased muscle tone associated with spasticity.

In specific embodiments, the patch, when used as intended, treats muscle spasticity.

In specific embodiments, the patch, when used as intended, treats muscle spasticity, including in conditions such as multiple sclerosis and spinal cord injury.

In specific embodiments, the patch, when used as intended, treats chronic neck and lower back pain.

In specific embodiments, the patch, when used as intended, treats regional musculoskeletal pain syndromes.

In specific embodiments, the patch, when used as intended, treats migraines (including analgesic rebound headaches).

In specific embodiments, the patch, when used as intended, treats fibromyalgia.

In specific embodiments, the patch, when used as intended, treats refractory insomnia in spastic quadriplegic patients.

In specific embodiments, the patch, when used as intended, treats at least one of chronic neck and lower back pain, regional musculoskeletal pain syndromes, migraines (including analgesic rebound headaches), fibromyalgia, and refractory insomnia in spastic quadriplegic patients.

In specific embodiments, the patch, when used as intended, for the management during opioid tapering.

In specific embodiments, the patch, when used as intended, as part of an anticonvulsant regimen.

In specific embodiments, the patch, when used as intended, is topically applied to the skin.

In specific embodiments, the patch, when used as intended, is applied to the skin and is followed by titrating the dose by applying successive transdermal patches at intervals ranging from 24 hours to 96 hours, to achieve optimal therapeutic effect with minimized side effects.

In specific embodiments, the patch, when used as intended, is adapted to deliver an effective amount of tizanidine base over a period of 24 to 48 hours.

In specific embodiments, the patch, when used as intended, reduces muscle tone and/or alleviates the symptoms of spasticity.

In specific embodiments, the patch, when used as intended, reduces muscle tone and/or alleviates the symptoms of spasticity, in patients who are unable or unwilling to swallow oral medication or in whom oral therapy is not well tolerated.

In specific embodiments, the patch, when used as intended, provides relief from spasticity during the nighttime or circadian periods, providing improved patient adherence compared to immediate-release oral regimens.

In specific embodiments, the patch is administered to patients who are unable or unwilling to swallow oral medication, or in whom oral therapy is not well tolerated.

In specific embodiments, the patch is administered to patients who are unable or unwilling to swallow oral medication.

In specific embodiments, the patch is administered to patients whom oral therapy is not well tolerated.

In specific embodiments, the patch is administered to patients who have demonstrated intolerance to oral tizanidine therapy.

In specific embodiments, the transdermal patch is applied to the skin and left in place for at least 24 hours during a course of therapy.

In specific embodiments, the transdermal patch is applied to the skin and left in place for at least 48 hours during a course of therapy.

In specific embodiments, the transdermal patch is applied to the skin and left in place for 24-96 hours during a course of therapy.

In specific embodiments, the transdermal patch is applied to the skin and left in place for 24-48 hours during a course of therapy.

In specific embodiments, the patch, when used as intended, maintains a steady-state plasma concentration of tizanidine base, wherein the maximum observed plasma concentration is less than the Cmax observed following an equivalent oral dose.

In specific embodiments, the patch, when used as intended, reduces or eliminates gastrointestinal side effects relative to oral administration of tizanidine, specifically abdominal pain and diarrhea.

In specific embodiments, the patch, when used as intended, avoids pharmacokinetic variability associated with the fed or unfed state of the subject, and delivers consistent therapeutic effect regardless of recent food intake.

In specific embodiments, the patch, when used as intended, the onset of therapeutic effect occurs at least about 2 hours after patch application.

In specific embodiments, the patch, when used as intended, the onset of therapeutic effect occurs up to about 8 hours after patch application.

In specific embodiments, the patch, when used as intended, the onset of therapeutic effect occurs between about 2 and about 8 hours after patch application.

In specific embodiments, when the patch is used as intended, the total amount of tizanidine base delivered does not exceed the equivalent of 36 mg per 24 hours.

In specific embodiments, when the patch is used as intended, the method bypasses hepatic first-pass metabolism to achieve systemic tizanidine bioavailability, at lower delivered dose compared to oral therapy, with improved safety.

In specific embodiments, when the patch is used as intended, achieves steady and sustained delivery, avoiding first-pass hepatic metabolism, and reducing gastrointestinal side effects relative to oral dosing.

In specific embodiments, when the patch is used as intended, achieves time to onset approximately 4 hours.

In specific embodiments, when the patch is used as intended, is suitable for once-daily or twice-weekly administration.

In specific embodiments, when the patch is used as intended, achieves steady and sustained delivery, avoiding first-pass hepatic metabolism, and reducing gastrointestinal side effects relative to oral dosing, with time to onset approximately 4 hours and duration suitable for once-daily or twice-weekly administration.

In specific embodiments, when the patch is used as intended, provides a steady-state plasma concentration of tizanidine base, with a Cmax at least 10% lower than that observed in oral dosing, and a lower incidence of abdominal pain and diarrhea relative to oral administration.

In specific embodiments, when the patch is used as intended, is suitable for patients requiring flexible or titratable transdermal dosing.

In specific embodiments, when the patch is used as intended, provides a steady-state plasma concentration of tizanidine base, with a Cmax at least 10% lower than that observed in oral dosing, and a lower incidence of abdominal pain and diarrhea relative to oral administration, said method suitable for patients requiring flexible or titratable transdermal dosing.

In specific embodiments, the patch, when used as intended, includes a titration regimen.

In specific embodiments, the patch, when used as intended, includes a titration regimen that includes starting dose protocols, sequencing, adjustment, and stopping criteria for inadequate effect or adverse events.

In specific embodiments, the methods of manufacturing the patch include visual particulate inspection, and second-person verification, before coating.

In specific embodiments, the methods of manufacturing the patch include accept/reject criteria in the event of batch out-of-spec findings (OOS).

In specific embodiments, when the kit includes: at least one transdermal patch described herein; separately packaged with the at least one patch, instructions for use directing application of the patch to the skin to treat increased muscle tone associated with spasticity, and provide titratable dosing; and further including at least one additional component selected from: (a) individually sealed pouches for each patch, (b) a child-resistant package, (c) a calendar or dosing schedule aid, (d) a disposal pouch for used patches, (e) a titration card or dose adjustment guide, (f) a desiccant or moisture-absorbing element, (g) a temperature indicator, (h) a QR code or web address for digital instructions, (i) multilingual instructions, and (j) a sterile wipe or pad for skin preparation.

In specific embodiments, when the kit includes: at least one transdermal patch described herein; separately packaged with the at least one patch, instructions for use directing application of the patch to the skin to treat increased muscle tone associated with spasticity, provide titratable dosing, and maintain reduced gastrointestinal side effects relative to oral tizanidine therapy; and further including at least one additional component selected from: (a) individually sealed pouches for each patch, (b) a child-resistant package, (c) a calendar or dosing schedule aid, (d) a disposal pouch for used patches, (e) a titration card or dose adjustment guide, (f) a desiccant or moisture-absorbing element, (g) a temperature indicator, (h) a QR code or web address for digital instructions, (i) multilingual instructions, and (j) a sterile wipe or pad for skin preparation.

In specific embodiments, each patch is provided in an individual sealed pouch within the kit.

In specific embodiments, the kit includes a child-resistant outer package.

In specific embodiments, the kit includes a calendar or marked packaging to assist with adherence to a dosing schedule.

In specific embodiments, the kit includes a calendar or schedule aid to assist with adherence to a dosing schedule.

In specific embodiments, the kit includes a disposal pouch or container for used patches.

In specific embodiments, the kit includes a titration card or dosing schedule to facilitate dose adjustment.

In specific embodiments, the kit includes a desiccant or moisture-absorbing element within the kit packaging.

In specific embodiments, the kit includes a storage temperature indicator.

In specific embodiments, the kit includes a QR code or web address linking to digital instructions or adherence support resources.

In specific embodiments, the kit includes digital adherence support via QR code, with associated software/app logic.

In specific embodiments, the kit includes written instructions provided in at least two different languages.

In specific embodiments, the kit includes a sterile wipe or pad for skin preparation before application.

In specific embodiments, the kit includes written and visual instructions to ensure correct patch application and removal, safe disposal, and guidance for dose titration.

In specific embodiments, the kit includes specialty packaging for disposal, color/shape markers for titration steps, skin cleansing wipes for improved adherence (with instructions for groups with excessive sweating or dermatological issues).

In specific embodiments, when the kit includes a disposal pouch for used patches, titration/dosing card, desiccant or temperature indicators, and scored release liner geometry.

In specific embodiments, when the kit includes a disposal pouch for used patches.

In specific embodiments, when the kit includes a titration/dosing card.

In specific embodiments, when the kit includes a desiccant or temperature indicators.

In specific embodiments, when the kit includes a scored release liner geometry.

The following examples are provided to further illustrate certain embodiments of the invention and are not intended to limit the scope thereof. Various modifications and variations will be apparent to those skilled in the art in light of the present disclosure, and such modifications and variations are intended to be included within the scope of the appended claims. All parts and percentages are by weight unless otherwise specified. The invention is not intended to be limited to the specific formulations, methods, or conditions described in these examples, and reference to specific embodiments is for purposes of illustration only.

### EXAMPLES

### Example 1: Solubility Screening and Solvent Compatibility Studies

A series of studies were conducted to evaluate the solubility characteristics of tizanidine base in a variety of solvents relevant to transdermal drug delivery system (TDS) formulations, and to assess solvent compatibility for subsequent adhesive and patch manufacturing steps. The objective was to identify suitable solvent systems for efficient drug dispersion during manufacture of an acrylic adhesive-based monolithic matrix patch.

### 1. Experimental Methodology:

Approximately 1 mg of tizanidine base (either micronized or unground) was added to individual glass test tubes. The solvents were selected from among those compatible with acrylic adhesives and previously reported for TDS manufacture, including ethyl acetate, methanol, ethanol (200P), dimethyl sulfoxide (DMSO), acetone, heptane, diethylene glycol (DEG), mineral oil, Tween 80, Transcutol, and lemongrass oil. Incremental additions of each solvent were made (1 mL increments up to a maximum of 5 mL). The mixtures were held at room temperature (ca. 20-25° C.), and observations—including solubilization, phase separation, or color change—were made at baseline and after 24 hours. All observations and data referenced herein pertain to both coarse and micronized forms unless otherwise noted.

### 2. Results:

Tizanidine base demonstrated minimal solubility in all solvents tested, except for slight solubility upon standing for 24 hours in diethylene glycol, ethyl acetate, and acetone. Notably, color changes in acetone and Transcutol samples (from yellow to dark yellow) suggest potential chemical interaction or degradation. The complete results are tabulated below:

| | Initial | After | Color after |
|---|---|---|---|
| Solvent | Observation | 24 hours | 24 hours |
| Light Mineral Oil | none | none | Yellow |
| Tween 80 | none | none | Yellow |
| Diethylene Glycol | none | slight | Yellow |
| Transcutol | none | none | Dark yellow |
| Methanol | none | none | Yellow |
| Ethyl Acetate | none | slight | Yellow |
| Lemongrass Oil | none | none | Yellow |
| Acetone | none | slight | Dark yellow |
| Heptane | none | none | Yellow |
| 200P Ethanol | none | none | Yellow |

### 3. Solvent Selection Rationale:

Ethyl acetate and ethanol were selected as preferred solvents, consistent with selection criteria detailed in the provisionals, due to (a) their proven compatibility with the chosen acrylic adhesives and (b) their ability to provide optimal dispersibility of tizanidine base as a suspension during manufacturing. Both solvents are Category 3 per USP <467> residual solvent requirements, supporting regulatory compliance.

### 4. Stability and Impurity Testing:

Subsequent testing by HPLC was performed to evaluate potential formation of impurities from solvent interaction. Acetone-containing samples produced a significant impurity peak, leading to their exclusion from all formulations. No significant process-related impurities were detected by HPLC in samples utilizing ethyl acetate or ethanol, in contrast to acetone.

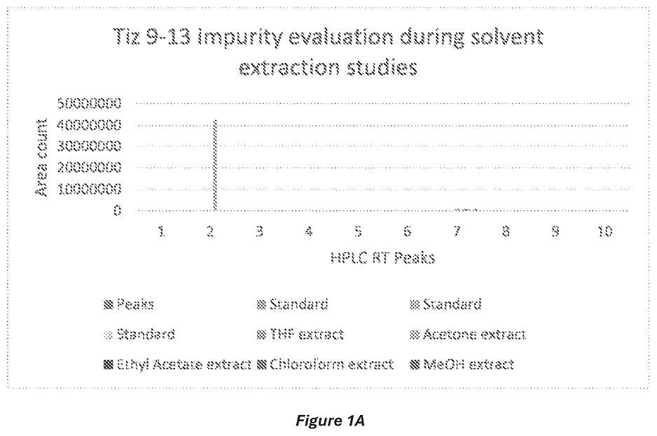

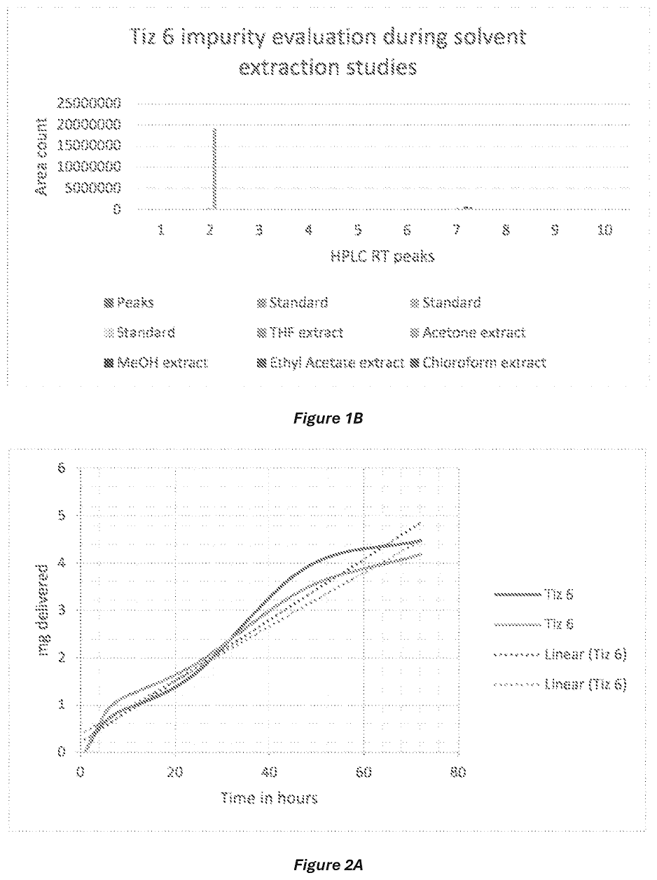

FIGS. 1A and 1B present HPLC chromatograms documenting impurity formation during solvent extraction studies. Specifically, these figures demonstrate that acetone use leads to the formation of prominent impurity peaks, supporting the exclusion of acetone from all product formulations and confirming the stability of tizanidine base in ethyl acetate and ethanol.

### Example 2: Adhesive Selection and Performance Screening

A series of screening studies were performed to identify suitable pressure-sensitive adhesives (PSAs) for use in the tizanidine base transdermal drug delivery patch, with emphasis on achieving optimal drug release, adhesion, and compatibility with the selected solvents and enhancers. Adhesives were tested for initial tack, shear, peel strength, compatibility with the tizanidine base, and impact on drug delivery performance.

### 1. Adhesive Candidates and Screening Parameters:

Commercial acrylic adhesives from the Duro-Tak® (Henkel) family, including grades 2074, 4287, 2516, GMS 788, and 2051, were evaluated. Initial screening encompassed the following attributes:
Tack and peel (adhesion to skin and liner)
Shear strength (ability to remain adhered during wear)
Compatibility with tizanidine base (chemical stability, no formation of impurities or degradation products as confirmed by HPLC)
Ability to form a homogeneous monolithic matrix with the insoluble tizanidine base and penetration enhancers
Maintenance of consistent release and physical characteristics under the manufacturing conditions

### 2. Results of Adhesive Screening:

A summary of observations for each adhesive is provided below:

| Duro-tak | |
|---|---|
| Adhesive | Observation |
| 2074 | Very low tack |
| 4287 | Very low tack |
| 2516 | Good adhesion |
| GMS 788 | Good tack, no drug delivery |
| 2051 | Good physical characteristics |
| | and delivery |

Duro-Tak 2516 exhibited high tack, shear, and peel properties and supported effective drug delivery.
Duro-Tak 2051 had high tack and peel but lower shear compared to 2516; its physical properties were advantageous after optimization with enhancers.
Adhesives 2074 and 4287 were eliminated early due to inadequate tack. GMS 788 provided good tack but failed to deliver tizanidine base effectively, as confirmed by dissolution/permeation analyses.

Both 2051 (with a —COOH functional group) and 2516 (with a —OH functional group) were selected for further study, including performance in combination with penetration enhancers (K29/32 povidone, Diethylene Glycol). Both adhesives supported stable incorporation of tizanidine base (without degradation or significant API loss, as confirmed by HPLC at multiple timepoints), with HPLC verification of drug stability and minimal impurity formation during processing or storage.

### 3. Impact of Adhesives on Drug Performance:

Formation of monolithic patches using either 2051 or 2516, together with enhancers, resulted in robust, reproducible patches with consistent coat thickness and content uniformity (±5% across the patch). Both adhesives provided suitable mechanical and wear properties for transdermal application.

### Example 3: Formulation Drug Load and Particle Size Optimization

A series of studies were conducted to optimize both the drug load and active particle size in the tizanidine base transdermal patch, with the goals of maximizing delivery efficiency, achieving content uniformity, and ensuring robust manufacturability.

### 1. Drug Load Range and Evaluation:

Candidate formulations were prepared with tizanidine base incorporated at varying loads, with the concentration expressed as % drug on solids basis within the adhesive matrix. Testing included drug loads between 8% and 16% (solids basis). Formulations were screened for content uniformity, patch integrity, drug release rate, and manufacturability. Loads below 8% of drug (solids basis) resulted in inadequate cumulative delivery, while exceeding 16% detrimentally affected coat integrity, uniformity, and mechanical properties.
The optimal range was determined to be about 12-15% by solids, yielding consistent performance with batch-to-batch content uniformity.
Each patch exhibited±5% content uniformity, with a typical 5 cm² sample yielding a target coat weight of approximately 0.045 grams.
Patches delivered about 3 mg±10% per 5 cm², with ±5% content uniformity documented by batch testing.

### 2. Particle Size Reduction of Tizanidine Base:

The tizanidine base active was evaluated as coarse crystal and after manual or supplier micronization.
Coarse (unchopped or unground) crystals were observed at sizes up to 0.030″ (~750 m), while ground/micronized samples demonstrated particle sizes less than 30 μm D90 as determined by laser diffraction.
Ground/micronized material displayed markedly superior dispersion properties in the adhesive, provided improved skin permeation, and showed a more linear and sustained dissolution profile compared to unground material.
Use of micronized tizanidine base allowed for more homogeneous suspension in the coating solution and enabled reduction in minimum effective patch size due to better dissolution kinetics.
Supplier-provided micronized tizanidine was preferred for manufacturing scale-up due to batch consistency and reproducibility.

### 3. Representative Formulation Data:

| | GSP413 | | GSP6 | |
|---|---|---|---|---|
| Ingredient | Amounts (g, %) | | Amounts (g, %) | |
| Adhesive (2051/2516) | 10 | (62.5/61.7%) | 10 | (61.7%) |
| Ethanol | 0 | (0%) | 3.5 | (21.6%) |
| Ethyl Acetate | 4 | (25%) | 0 | (0%) |
| K29/32 | 0.5 | (3.13%) | 0.6 | (3.70%) |
| Tizanidine | 1 | (6.25%) | 0.7 | (4.32%) |
| DEG | 0.5 | (3.13%) | 1.4 | (8.64%) |

| | | |
|---|---|---|
| Total Solids | 7.15 | 6.85 |
| % Drug solids based | 13.99% | 10.22% |

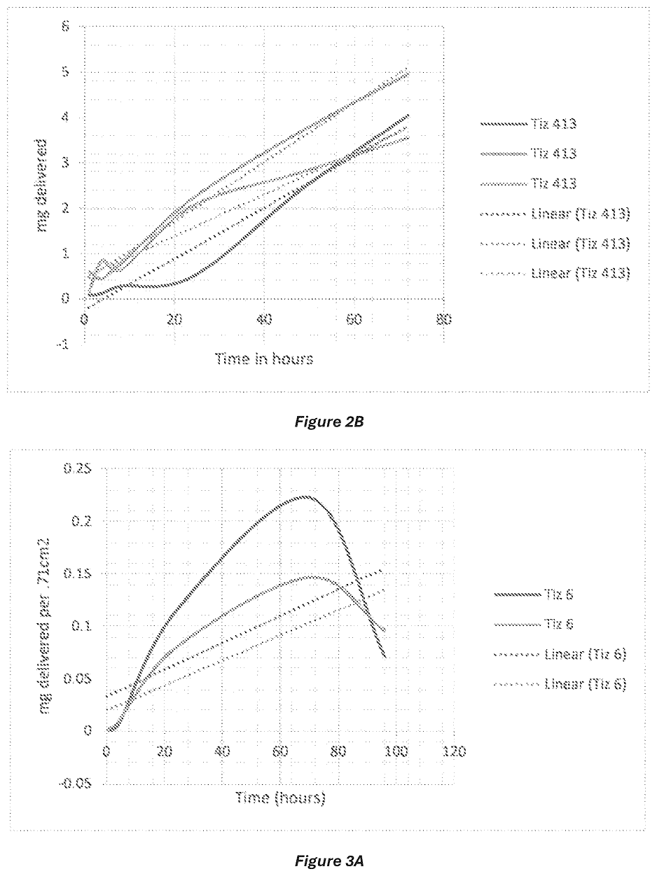

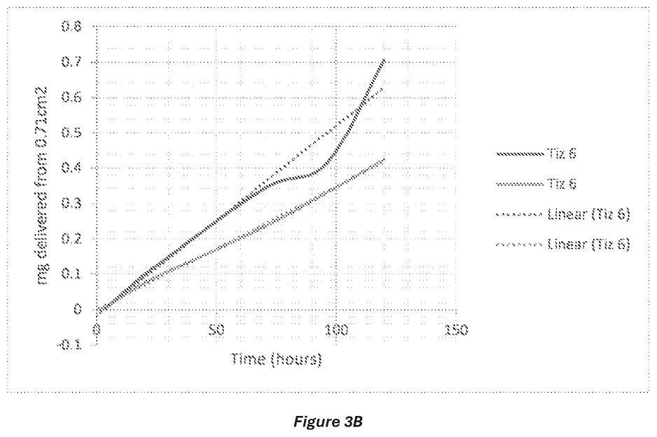

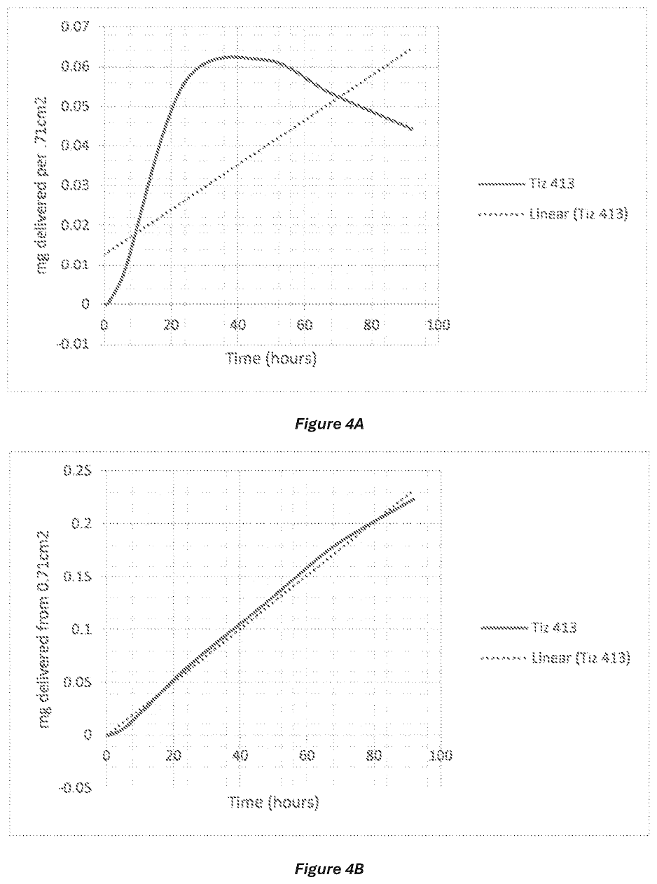

FIGS. 2A and 2B show dissolution profiles (cumulative drug delivered over 72 hours) for GSP6 and GSP413 patch formulations respectively, validating sustained and uniform delivery. FIGS. 3A, 3B, 4A, and 4B depict skin permeation rate and cumulative profiles from both patch types, demonstrating improved flux and delivery performance with micronized tizanidine base.

### Example 4: Formulation Development of Tizanidine Base Transdermal Systems

A series of formulation development studies were performed to identify optimal component selection, blending methods, and process parameters for the preparation of tizanidine base transdermal drug delivery systems. The primary objectives were to maximize skin permeation, maintain content uniformity, and ensure scalable manufacturability.

### 1. Component Selection:

Formulation development began with the selection of pressure-sensitive adhesives (PSAs) based on acrylic copolymers, such as Duro-Tak® 2051 and 2516 (Henkel), penetration enhancers, and solvents. Henkel Duro-Tak® acrylic adhesives were screened for adhesion, tack, and compatibility with tizanidine base. Duro-Tak 2051 (with —COH functionality) and Duro-Tak 2516 (with —OH functionality) were identified as most suitable, based on balanced peel, shear, and tack, as well as chemical stability of the active drug. The penetrative enhancers polyvinylpyrrolidone (K29/32) and diethylene glycol (DEG) were included to improve skin flux and delivery.

### 2. Formulation Method and Suspension Preparation:

Due to poor solubility of tizanidine base in most pharmaceutically acceptable solvents, the drug was incorporated as a micronized particulate suspension. Ethanol and/or ethyl acetate were chosen as primary solvents for compatibility with both the drug and adhesive. Order of addition was optimized such that solvent, enhancer(s), and tizanidine base were premixed, followed by careful addition and mixing of the adhesive to minimize agglomeration and ensure batch content uniformity. Homogenization was performed (2-5 minutes at ~10,000 rpm with a 7-blade shear head) to ensure uniform drug dispersion. Continuous mixing and protection from light were maintained throughout processing.

### 3. Optimization of Coating and Laminating Parameters:

The coating solution was applied by knife-over-plate onto a fluoropolymer-coated polyester release liner (3M ScotchPak 9744 or 1022), target coat thickness 0.013 inches (33 μm). Coated films were dried in a staged oven (70-75° C. for 5 minutes, then 90-100° C. for 3 minutes), then immediately laminated to a backing film (either 3M ScotchPak 9738—aluminized occlusive film, or 1012—clear flexible film).

### 4. Representative Final Formulations:

| | GSP413 | GSP6 |
|---|---|---|
| Ingredient | (% w/w) | (% w/w) |
| Adhesive | 62.50 (2051) | 61.73 (2516) |
| Ethanol | 0 | 21.60 |
| Ethyl Acetate | 25.00 | 0 |
| K29/32 | 3.13 | 3.70 |
| Tizanidine Base | 6.25 | 4.32 |
| DEG | 3.13 | 8.64 |
| Drug solids (% base) | 13.99 | 10.22 |

All formulations demonstrated process robustness, batch-to-batch uniformity (±5%), and suitable coat weights and adhesion.

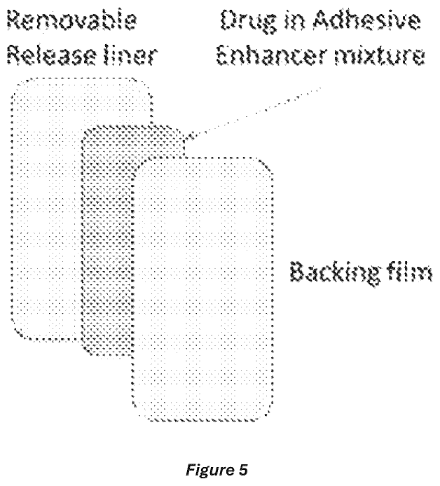

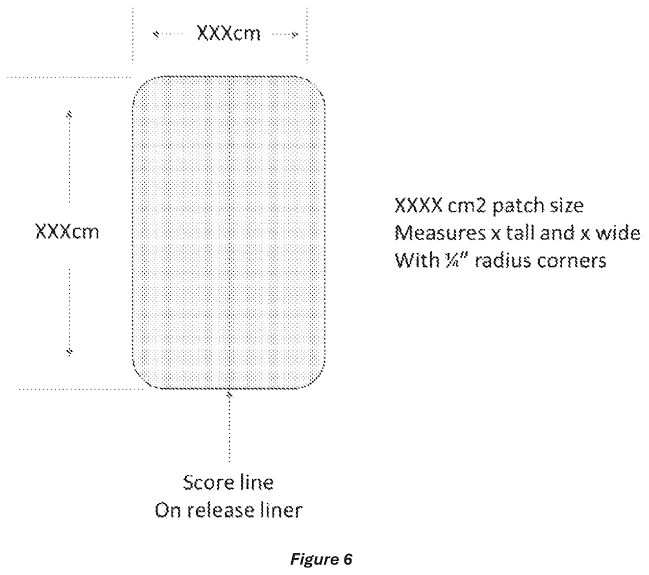

FIGS. 5 and 6 clarify patch construction. FIG. 5 presents an exploded schematic, depicting the adhesive matrix, release liner, backing film, and enhancer location for robust assembly. FIG. 6 provides a top-down view showing patch layout and scoring, relevant for quality and application control.

### Example 5: Use of Various Penetration Enhancers with Acrylic Adhesives

Multiple formulation studies were conducted to evaluate the effectiveness of various penetration enhancers in combination with acrylic pressure-sensitive adhesives. Penetration enhancers were selected to maximize skin permeation, modify release kinetics, and maintain stability when incorporated into monolithic patch matrices.

### 1. Penetration Enhancer Selection and Screening

Primary penetration enhancers (materials incorporated to increase transdermal absorption by modifying skin permeability) assessed included polyvinylpyrrolidone (povidone, K29/32 and K90) and diethylene glycol (DEG), both compatible with Henkel Duro-Tak® acrylic adhesives. Additional candidates such as light mineral oil, Tween 80, lemon grass oil, and Transcutol were initially explored but did not yield significant improvements in solubility or permeation for tizanidine base. Screening indicated that only diethylene glycol yielded minor solubility and significant permeation enhancement.

### 2. Matrix Testing and Comparative Deliverability

Matrix formulations were created using acrylic adhesives (Duro-Tak 2516 and 2051), with combinations and concentrations of povidone and DEG varied to assess impact on drug flux and patch performance. Representative test data is summarized below:

| Ingredients | Tiz-2b | Tiz-2c | Tiz-2d | Tiz-6b | Tiz-6c | Tiz-6d |
|---|---|---|---|---|---|---|
| Adhesive | 5 g | 5 g | 5 g | 5 g | 5 g | 5 g |
| MeOH | 2.1 g | 2.1 g | 2.1 g | 2.1 g | 2.1 g | 2.1 g |
| Ethyl acetate | 1 g | 1 g | 1 g | 1 g | 1 g | 1 g |
| K29/32 (4%) | — | 0.25 g | — | 0.25 g | — | — |
| K90 | — | — | 0.25 g | — | 0.25 g | — |
| Tizanidine | 0.25 g | 0.25 g | 0.25 g | 0.5 g | 0.5 g | 0.5 g |
| DEG/Enhancer | 0.25 g | 0.5 g | 0.75 g | 0.5 g | 0.75 g | 1 g |

Incorporation of K29/32 and DEG at varying concentrations increased tizanidine base flux from the matrix, as evidenced by in vitro skin permeation and dissolution studies. Patches incorporating both povidone and DEG provided the highest and most consistent delivery, with drug loading ranging from about 8%-16% solids basis and cumulative delivery matching oral reference standards.

FIGS. 2A/2B show enhancer-driven release profiles; FIGS. 3A/3B and 4A/4B depict skin permeation for various enhancer matrices, supporting selection for optimal flux.

### Example 6: Dissolution Profile Studies of Tizanidine Patches

Dissolution profile studies were conducted to evaluate the cumulative and time-dependent release of tizanidine base from various transdermal patch formulations. These studies enabled assessment of sustained drug delivery, release kinetics, and batch uniformity between alternative formulations prepared using distinct adhesives and penetration enhancers.

### 1. Formulation Composition

GSP6 formulation: Acrylic adhesive (Duro-Tak 2516), ethanol solvent, povidone K29/32, diethylene glycol, and tizanidine base (drug load: 10.22% solids basis).
GSP413 formulation: Acrylic adhesive (Duro-Tak 2051), ethyl acetate solvent, povidone K29/32, diethylene glycol, and tizanidine base (drug load: 13.99% solids basis).

### 2. Experimental Methods

Representative patches (5 cm² samples) were incubated in 0.1 M sodium phosphate buffer (pH 7.2) at room temperature. The buffer solution was sampled over a 72-hour period, and tizanidine concentrations were quantified using modified USP analytical methods. Duplicate and triplicate analyses were performed for each patch batch.

### 3. Results and Analysis

Both formulations demonstrated linear and sustained cumulative release of tizanidine base across the test period, with GSP413 providing relatively higher cumulative delivery due to increased drug loading and concentration of enhancers. Each patch delivered approximately 3 mg±10% tizanidine over 48 hours, matching reference dissolution criteria for steady-state transdermal administration. The profiles matched the target area under the concentration-time curve (AUC) for oral reference, providing steady-state delivery and reduced Cmax compared to oral administration. For each dissolution test, n≥2 patches per batch.

### 4. Recommended Visuals for Inclusion

For clarity and completeness, the following figures should be included:
FIG. 2A: Dissolution profile (mg/time) for GSP6 formulation (shows linear cumulative delivery from 5 cm² patch over 72 hours).
FIG. 2B: Dissolution profile (mg/time) for GSP413 formulation (shows linear cumulative delivery from 5 cm² patch over 72 hours).

FIGS. 2A and 2B directly illustrate cumulative drug release profiles for GSP6 and GSP413 patches, respectively. These figures provide graphical evidence supporting linear, sustained, and batch-uniform delivery over 72 hours in buffer media simulating physiological skin conditions.

### Example 7: Skin Permeation Studies Using Human and Animal Models

Skin permeation studies were conducted to assess the transdermal delivery of tizanidine base from prototype patches, evaluating both the rate and cumulative amount of drug delivered through human and animal skin ex vivo. These studies were fundamental for establishing bioequivalence to oral products, validating efficacy, and optimizing dosage forms.

FIGS. 3A and 3B show GSP6 patch skin permeation delivery as both rate (mg delivered per time) and cumulative amount (72-hour total). FIGS. 4A and 4B perform the same for GSP413. These figures confirm continuous, reproducible permeation rates that match pharmacokinetic targets for oral bioequivalence

### 1. Experimental Methods

Representative patches (0.71 cm²) were evaluated on human cadaver or validated animal skin in Franz diffusion cells equipped with neutral pH sodium phosphate buffer. Receiver fluid was sampled periodically over 72 hours and analyzed for tizanidine concentration (via HPLC).

### 2. Key Results

Both GSP6 (using Duro-Tak 2516 adhesive) and GSP413 (using Duro-Tak 2051 adhesive) formulations demonstrated continuous, linear cumulative permeation profiles, with replication across multiple samples confirming reliability.
GSP6 exhibited higher cumulative delivery relative to GSP413, attributed to its unique composition and drug load.
The permeation rates and total delivery were consistent with requirements for matching oral area under the concentration-time curve (AUC), but provided diminished Cmax and reduced GI side effects.

FIGS. 3A and 5B show GSP6 patch skin permeation delivery as both rate (mg delivered per time) and cumulative amount (72-hour total). FIGS. 4A and 4B perform the same for GSP413. These figures confirm continuous, reproducible permeation rates that match pharmacokinetic targets for oral bioequivalence.

### Example 8: Comparative Pharmacokinetic Studies (Patch Vs. Oral)

Comparative pharmacokinetic studies were performed to evaluate systemic delivery of tizanidine base from prototype transdermal patches versus conventional oral dosage forms (tablet or capsule). These studies included skin permeation experiments in animal models, dissolution studies, and direct comparative analysis of drug plasma profiles.

### 1. Experimental Methods

Rodent models and validated ex vivo human skin permeation systems were used to collect plasma and receiver compartment samples following administration of both the transdermal patch and oral tizanidine formulations. Blood serum samples were analyzed by HPLC to quantify tizanidine and metabolite levels over time. For the oral dosage form, subjects were dosed at 2-4 mg, with pharmacokinetic parameters adjusted for known hepatic first-pass metabolism.

### 2. Key Findings

Bioequivalence Achieved: The transdermal patch matched or approximated the area under the curve (AUC) of the oral product over a 24-48 hour period, demonstrating comparable overall drug delivery.
Reduced Cmax: Peak plasma concentrations (Cmax) were consistently lower for the patch than for oral dosing, achieving a steady-state profile ideal for minimizing Cmax-related side effects and avoiding oral spikes.
Onset and Duration: The patch exhibited a slower onset (approximately 4 hours) compared to oral dosing (about 30 minutes unfed, 2 hours fed), but maintained consistent and sustained therapeutic plasma levels thereafter.
Fed/Unfed Variability Eliminated: Patch delivery circumvented fed/unfed variability, providing uniform systemic exposure regardless of food intake.
Reduced GI Side Effects: The patch system avoided gastrointestinal adverse effects associated with oral tizanidine (e.g., abdominal pain, diarrhea), further supporting its safety advantage.

FIGS. 2A and 2B are especially relevant, showcasing the dissolution over time for patch designs. The displayed data supports the claim that patch delivery matches the area under the curve (AUC) of oral tizanidine, with reduced Cmax and absence of GI side effects. Summary graphs in the text further visualize oral versus patch pharmacokinetics.

### Example 9: Fed/Unfed Pharmacokinetic Equivalence Testing

Comparative pharmacokinetic studies were conducted to assess the impact of fed versus unfed (fasted) states on the absorption and systemic exposure of tizanidine delivered via both the oral and transdermal patch formulations. These studies are significant to establish whether the transdermal dosage form demonstrates pharmacokinetic equivalence and reduced variability relative to oral administration, which is known to be affected by recent food intake.

### 1. Experimental Methods

A crossover pharmacokinetic study design was employed in which subjects received oral tizanidine (tablet or capsule) in both the fasted and fed states, as well as the prototype transdermal patch, under controlled conditions. Blood samples were collected at defined intervals and analyzed for tizanidine concentrations using validated HPLC assays. The primary endpoints assessed included area under the curve (AUC), Cmax, Tmax, and pharmacokinetic variability between conditions.

### 2. Key Findings

Oral Administration: Oral tizanidine exhibited significant fed/unfed variability. In the unfed state, peak concentrations (Cmax) were achieved rapidly (about 30 minutes), whereas administration with food increased Cmax by ~30% (tablets) or decreased it by ~20% (capsules), and delayed Tmax to as much as 3 hours. Food also increased overall absorption for both dosage forms, with the extent of the effect differing by formulation.
Transdermal Patch: In contrast, the transdermal patch provided consistent systemic delivery and exposure, independent of feeding state. Application of the patch resulted in a steady increase in plasma tizanidine concentrations over several hours (onset ~4 hours), maintained at therapeutic levels without the Cmax spikes or variability observed after oral dosing in different nutritional states.
Clinical and Formulation Implications: The reduction or elimination of fed/unfed variability with the patch supports The elimination of fed/unfed variability with the patch supports robust therapeutic performance and predictable systemic exposure for broad patient populations, independent of meal timing. This characteristic is particularly advantageous for populations with variable eating habits or at risk for adverse events from Cmax-related effects.

FIGS. 2A and 2B are cited as visual evidence for placebo and active patch dissolution consistency, independent of feeding state. These demonstrate that the patch provides steady drug delivery unaffected by oral absorption variability tied to food intake.

### Example 10: Evaluation of Release Liners and Backing Films

Comprehensive liner and backing film screening studies were conducted to determine optimal materials for the transdermal tizanidine base patch. Selection criteria included clean release functionality, compatibility with adhesive and coating solution, physical strength, occlusivity, and drug delivery performance. Comparative testing and analytical evaluation were performed utilizing multiple materials.

### 1. Materials and Methods

Release Liners: 3M® ScotchPak 9744 (fluoropolymer-coated polyester, caliper 2.91 mils/74 μm, clear) and ScotchPak 1022 (fluoropolymer-coated polyester, identical caliper and characteristics) were evaluated for ease of release, coating uniformity, and residual release agent levels.
Backing Films: 3M® ScotchPak 1012 (clear matte polyester with EVA copolymer) and ScotchPak 9738 (tan polyethylene-aluminum vapor-coated polyester with occlusive tie layer) were modified and tested for occlusivity (MVTR <0.3 g/m²/24 hr for 9738), physical robustness, and performance under wear conditions.
Methods included visual inspection, peel and adhesion performance measurement, tensile testing, moisture vapor transmission rate analysis, and assessment of impacts on drug release profile and color compatibility.

### 2. Results and Observations

Both 9744 and 1022 liners provided clean, reproducible release and were fully compatible with both GSP6 and GSP413 patch formulations; no difference in drug delivery or surface adhesion properties was observed between the two.
Backing film 1012 gave transparent, flexible support for standard studies; backing film 9738, offering an occlusive barrier and aesthetic skin-tone coloration, was preferred for commercial designs requiring enhanced environmental insulation or patient masking.
No measurable difference in delivery rate, color intensity, or mechanical integrity was found between liner or backing film candidates during in vitro and skin permeation analysis, supporting sourcing flexibility.

FIGS. 6 and 12 are highlighted, as they depict the patch layout, showing the scoring of the release liner and the placement of the adhesive matrix with backing film. These visuals confirm correct patch geometry and handling for user application and removal, as well as compatibility of alternative liner/backing materials.

### Example 11: Impurity/Degradation Product Analysis by HPLC

All analytical methods were validated to USP and ICH Q3A (R2) guidelines for related substances.

Impurity formation and degradation product analysis were conducted on the tizanidine transdermal patch formulations using high-performance liquid chromatography (HPLC), in accordance with industry-standard analytical protocols. The objective was to assess the chemical stability of tizanidine base throughout manufacturing, storage, and delivery, and to ensure that impurity levels remained below pharmaceutically acceptable limits.

### 1. Analytical Methods

Patch samples and extracted actives were evaluated using validated HPLC methods with specific calibration for tizanidine base and known impurities.
Stress testing was performed on patches prepared using different solvent and adhesive systems, including exposure to acetone, ethyl acetate, methanol, tetrahydrofuran (THF), and chloroform, as well as blank controls.
Impurity identification included: butyl 4-hydroxybenzoate, 4-amino-5-chloro-2,1,3-benzothiadiazole, N-acetyltizanidine, and 1-acetylimidazolidine-2-thione, as provided by the tizanidine base supplier.

### 2. Results and Observations

HPLC chromatograms indicated that acetone exposure resulted in the formation of a large impurity peak not present in ethyl acetate, methanol, or blank samples, leading to exclusion of acetone from all manufacturing steps.
Both GSP6 and GSP413 formulations demonstrated low total impurity counts, with major and total impurities consistently below 0.1% by weight after processing and storage, meeting the target specification.
Extracted samples from test patches confirmed chemical stability and absence of significant degradation throughout coating, drying, laminating, packaging, and storage.

FIGS. 1A and 1B show impurity peaks in chromatograms obtained from solvent extraction studies. These figures document the chemical stability of the tizanidine base and impurity profile in final patch formulations, confirming robust control and process purity.

### Example 12: Content Uniformity and in-Process Quality Controls

Content uniformity and in-process quality controls were rigorously evaluated for all tizanidine transdermal patch formulations as disclosed in the cited provisional applications. The goal was to ensure each patch meets specifications for active drug distribution, weight, and physical performance, both during and after manufacture.

### 1. Analytical Methods and Procedures

Patches were sampled throughout production from the master roll at various positions (left, middle, right; beginning, middle, end) to assess weight, thickness, and drug content uniformity.
Drug content was measured by weighing 1-inch diameter cut samples, accounting for release liner and backing film, with total coat weight determined as:

Coat weight=Sample Weight−(Release Liner+Backing Film Weight)Coat weight=Sample Weight −(Release Liner+Backing Film Weight)
Coating thickness was measured via micrometer, adjusted by subtracting liner and backing film thickness from the total sample thickness.
Uniformity of tizanidine content within and between patches was assayed using high-performance liquid chromatography (HPLC), following destruction of the matrix and extraction of the active for analysis.

### 2. In-Process Controls

All raw materials were weighed and verified by a second individual.
Visual inspection for particulate and undissolved material was conducted at solution preparation and after coating.
Micrometer and feeler gauge measurements were used at both sides and the middle of the coating head to maintain consistent coat thickness across the film.
Samples taken at multiple points on each roll were checked for content uniformity, coat thickness, total weight, and absence of bubbles or streaks.
Testing for content uniformity required all samples to fall within +5% of the mean tizanidine content per patch, in line with the proposed product specifications.

### 3. Quality Control Specifications

Content uniformity: ±5% of nominal tizanidine content per patch.
Patch thickness: 0.008″±10%.
Coat thickness: 0.013″ (33 mm) ±10%.
Patch weight: 0.1250 grams±10% (5 cm² patch).
Coat weight: 0.0450 grams±10% (5 cm² patch).
Adhesion/pull/peel: Minimum 200 seconds tack at 6 grams, shear of ≥100 grams, and 90-degree peel strength of 100-250 grams force.
Dissolution: Delivers 3 mg±10% tizanidine over 48 hours, with uniform in vitro release profile.

### Example 13: Patient Titration and Dosing Regimen Studies

Patient titration and dosing regimen studies were performed to establish optimal approaches for safe and effective use of the tizanidine transdermal patch, ensuring therapeutic equivalence with oral delivery while accommodating interpatient variability and minimizing side effects.

### 1. Study Design

Objective: To define titratable transdermal dosing regimens for tizanidine that achieve effective reduction of muscle spasticity, mimic or improve upon oral dosing pharmacokinetics, and offer flexible adjustments based on clinical response.
Patch configurations: Patches were manufactured in multiple discrete sizes (e.g., 5 cm², 10 cm², 20 cm²) to provide incremental dosing, in line with clinical titration requirements.
Initial titration protocol: Patients began with the lowest patch size (e.g., 5 cm² delivering approximately 0.75-1 mg/24 hr) applied to a hairless area, with the patch left in place for 48 hours.
Titration adjustments: If adequate reduction in muscle tone was not achieved and side effects were tolerable, the dose was increased to the next patch size (e.g., 10 cm² delivering 1.5-2 mg/24 hr) at the next scheduled change. Titration continued at intervals of 48-96 hours, up to a maximum size (e.g., 20 cm² delivering target of 3-4 mg/24 hr), or until patient response plateaued.
Endpoints measured: Muscle tone reduction (Ashworth or equivalent scale), occurrence of adverse events (especially gastrointestinal), steady-state plasma drug concentration, and patient adherence.
Special populations: Titration protocols also considered subgroups with oral therapy intolerance, elderly or renally impaired subjects, and those with fed/unfed variability on oral therapy.

### 2. Results and Findings

The majority of participants reached satisfactory symptom control within 2-4 titration steps, with plasma concentrations remaining within the therapeutic window established for oral tizanidine therapy.
Adverse event incidence (notably GI side effects) was lower as compared to oral titration, reflecting the patch's avoidance of hepatic first pass and corresponding plasma Cmax spikes.
Steady-state was typically reached by the second or third patch application, per PK projections and limited patient blood sampling.
Patients reported improved adherence and satisfaction due to the less frequent patch change compared to multiple daily oral doses.
Dose adjustments were achieved in 48- to 96-hour increments, paralleling the flexibility provided in oral titration protocols.

FIG. 6 (top-down view), along with titration tables, serve to illustrate discrete patch sizes and dose increments available for titratable therapy. These visuals help clarify patient adjustment pathways and clinical protocols for individualized dosing.

### Example 14: Manufacturing Process and Order-of-Addition Optimization

A robust manufacturing process for the tizanidine base transdermal patch was developed to optimize content uniformity, minimize impurity formation, and provide scale flexibility for both lab and commercial production. The process sequence and order of ingredient addition were found to be significant to the final product's physical characteristics and drug delivery performance.

### 1. Process Overview

The manufacturing process includes four main steps: (1) preparation of the coating solution, (2) coating and drying, (3) laminating and die-cutting, and (4) packaging.

### A. Coating Solution Preparation

Sequential Addition (Significant Order): For the GSP6 formulation, ethanol, K29/32 (polyvinylpyrrolidone), diethylene glycol (DEG), and tizanidine base are first combined and stirred to achieve a uniform suspension. The acrylic adhesive (either Henkel Duro-tak 2516 or 2051) is then added last, followed by additional mixing under light-protected conditions.
Mixing Controls:

The mixture is homogenized for 2-5 minutes at high shear (approximately 10,000 rpm) to ensure even active pharmaceutical ingredient (API) dispersion. Continuous mixing is maintained to prevent phase separation; solutions are stored protected from light for up to 36 hours prior to coating.

### B. Coating and Drying

Application:

The thoroughly mixed suspension is coated onto a treated fluoropolymer-coated polyester film (3M ScotchPak 9744 or 1022) using a knife-over-plate or knife-over-roll mechanism, with thickness set via micrometric adjustment to 0.013″ (±10%).
Drying:

Coated films are dried in two stages: 5 minutes at 70-75° C., then 3 minutes at 90-100° C. Upon completion of drying, films are immediately allowed to cool to room temperature and then laminated to the backing film (e.g., 3M ScotchPak 9738 or 1012).

### C. Laminating and Die-Cutting

Lamination:

Backing film is nipped to the dried coated film with moderate, controlled pressure.
Die-Cutting:

Patches of defined area (e.g., 20 cm²) are cut with score lines in the release liner to facilitate easy removal. Product is inspected for uniform thickness and weight.

### D. Packaging

Primary Pouching:

Each patch is individually sealed in an aluminum-laminate pouch, heat sealed on all four sides to prevent moisture and light ingress.
Secondary Controls:

Lot numbers and expiry dates are embossed or printed on the pouch; pouches are boxed with dosing instructions and optional accessories.

### 2. Order-of-Addition Impact

Studies demonstrated that improper addition Out-of-sequence addition (e.g., incorporating adhesive before API and enhancers) resulted in clumping, poor drug dispersion, and reduced delivery consistency. The optimized order consistently yielded patches with <5% content variability and impurity levels <0.1% by HPLC at release and after 12 months ambient storage.

FIG. 5 (exploded view) and FIGS. 8, 9, 10 (perspective machine diagrams) explain order-of-addition, mixing, coating, drying, laminating, and packaging steps, and visually document scale-up from lab through pilot to commercial production. FIG. 6 confirms product geometry after lamination and die-cutting.

### Example 15: Stability Studies Under Ambient and Light-Protected Storage

All patches tested were in final (aluminum pouch) commercial packaging format when indicated.

Stability testing was performed to assess the retention of tizanidine base content, impurity profile, and delivery characteristics of the transdermal patch during storage under both ambient conditions and protection from light. These studies were carried out on production-representative patches fabricated according to the optimized GSP6 and GSP413 formulations.

### 1. Study Design and Procedures

Storage Conditions:

Patches were stored at ambient room temperature (20-25° C.), with test groups either shielded completely from light (stored in aluminum pouches) or exposed to typical room lighting, for intervals up to 12 months.
Sampling and Time Points: Analytical samples were withdrawn initially and at monthly intervals for up to 12 months.
Key Parameters Evaluated:
   Assay for tizanidine base content (by HPLC)
   Total and individual impurity levels (by HPLC)
   Dissolution profile and drug release characteristics
   Physical appearance, color, and integrality of the matrix and packaging

### 2. Results

Tizanidine Content:

Patches maintained drug content within ±5% of label claim after 12 months in both storage conditions, confirming excellent stability of the API in the matrix.
Impurity Profile:

Major and total impurity levels remained below 0.1% throughout storage, with no new degradant peaks observed in HPLC analysis under either light-protected or ambient conditions.
Dissolution and Delivery:

Drug release rates, as measured in in vitro dissolution studies, remained consistent with initial profiles at all time points, and content uniformity across the patch was maintained within ±5%.
Physical Appearance:

No macroscopic or microscopic changes (e.g., matrix discoloration, patch delamination, or pouch compromise) were detected. Patches stored in light-protective pouches retained color and appearance identical to those stored in darkness.

FIGS. 2A and 2B show the ongoing dissolution profile for patches stored under ambient and light-protected conditions, while FIGS. 1A and 3B depict impurity tracking over time. These figures collectively demonstrate retention of both delivery rate and chemical stability after prolonged storage.

### Example 16: Testing of Alternative Release Liner and Backing Compositions

Alternative release liner and backing film compositions were evaluated to determine their impact on the physical integrity, drug delivery performance, and manufacturability of tizanidine-base transdermal patches. Screened liner materials included 3M® ScotchPak 9744 and ScotchPak 1022 fluoropolymer-coated polyester films, while backing films 3M® ScotchPak 1012 (clear matte polyester and EVA copolymer) and ScotchPak 9738 (tan polyethylene and aluminum vapor-coated polyester) were tested for occlusive properties.

### 1. Methods

Patch Preparation

Standard GSP6 and GSP413 formulations were cast using an identical process, varying only the liner and/or backing composition. Each batch adhered to the typical thickness (0.013″±10%) and patch size specifications (5 cm² or 20 cm²).
Evaluated Parameters
   Efficiency of transfer from release liner to backing film
   Physical removal (“release”) characteristics of the coated liner
   Final patch adhesion, flexibility, durability, and occlusivity
   Effects on drug content uniformity and in vitro release profile

### 2. Results

Release Liner Performance

Both ScotchPak 9744 and 1022 liners enabled clean removal and transfer of the drug layer to backing films, exhibiting similar fluoropolymer surface characteristics and no significant effect on drug release or adhesion properties.
Backing Film Properties

ScotchPak 1012 (clear, flexible) and ScotchPak 9738 (tan, aluminum) backing films showed no difference in drug delivery rate or cumulative release. Occlusive (aluminum) backing further reduced moisture vapor transmission, as measured by MVTR tests (<1 g/m²/24 hr for 9738), supporting use for improved shelf-life and API stability. Durability, tack performance, and peel strength remained within predefined product specifications for both alternatives.

FIG. 5 is used to illustrate the spatial relationships and compatibility between different liner and backing film choices. FIG. 6 further details the scoring and configuration of the patch when assembled with these alternatives, confirming uniformity and interchangeability.

### Example 17: Process Scale-Up and Pilot Manufacturing Validation

A process scale-up and pilot manufacturing validation study was conducted to confirm robust translatability of laboratory-based methods for tizanidine-base transdermal patch preparation to pilot and commercial scale equipment and processes. Both main (GSP6) and backup (GSP413) formulations were evaluated.

### 1. Methods

Small-Scale Preparation:

Initial batch production was performed using a hand-draw down knife over glass plate on 3M® 9744 or 1022 release liners. Coat thickness and uniformity were confirmed by micrometer and feeler gauge measurements. Drying was carried out in standard laboratory convection ovens (staged at 70-75° C. and 90-100° C.), followed by manual lamination onto 9738 or 1012 backing films.
Pilot-Scale Production:

Formulations found suitable at small scale were prepared using a pilot coating machine featuring knife-over-plate mechanism and 2-stage dryer zones. Key parameters included adjustability of coat thickness, unwinding and rewinding tension for films, and accurate nip/lamination force control. Coating speeds ranged from 2-5 fpm, yielding latency periods in ovens of approximately 7 minutes. The final master roll of patch material was cooled, sampled for product integrity, and stored under ambient or shielded-from-light conditions.
Validation:

Representative placebo (adhesive matrix absent the active ingredient) and active patch lots were further validated on a commercial HED-manufactured coating line to benchmark pilot-scale results and assess scalability and reproducibility.

### 2. Results

Coat Thickness & Uniformity:

Across both small and pilot scale, consistent coat thicknesses of 0.013″ (33 mm) ±10% were achieved, as measured by micrometer and confirmed via in-process quality checks on both left/middle/right positions and start/end segments of master rolls.
Lamination & Adhesion:

Immediate lamination post-drying was practiced, preventing excessive tack variability and ensuring consistent adhesion and peel strength within specified ranges. Finished master rolls demonstrated stability for up to 15 months when shielded from light, with no increase in impurity levels observed over labeled shelf life.
Sampling & QA:

Samples cut during production consistently met criteria for patch thickness, coat weight (0.045 g/5 cm²±10%), drug load, and absence of physical anomalies (bubbles, streaks, delamination). Product integrity and drug content were confirmed by HPLC assay and in-process controls. Results demonstrated compliance with both in-process and finished product specifications

### Example 18: Packaging and Patch Kit Assembly Process

A packaging and kit assembly process was established to ensure integrity, stability, and user-friendly handling of the tizanidine transdermal patch product during distribution and use.

### 1. Packaging Procedures

Primary Packaging:

Each transdermal patch is die-cut to a rectangular shape with rounded edges (typically 5-20 cm²), retained on a scored release liner, and immediately placed into a pouch constructed from aluminum-faced packaging material. The pouch is heat-sealed on all four sides at temperatures ranging from 250-350° F. with a nominal dwell time of 1 second, ensuring an airtight and light-impermeable barrier for product protection.
Labeling and Identification:

The packaging pouch is embossed or printed with the lot number and may include additional information such as the product name and instructions, either directly printed, applied as a label, or pre-printed on the pouching material.
Kit Assembly:

Individually sealed pouches containing one patch each are grouped into labeled secondary cartons or kits, which may incorporate child-resistant features, titration or dosing guides, a calendar/schedule, and/or secondary disposal pouches. Additional optional components include desiccants, temperature indicators, and QR codes or web links for digital instructions and adherence support, as well as sterile wipes for skin preparation.
Quality Controls:

During packaging, samples are collected every 15 minutes to check seal integrity using burst testing. Placement of the patch within the pouch is inspected to confirm it has not moved into the seal area and that pouches are intact. Final shelf cartons are checked for proper assembly and labeling.
Storage:

Finished and pouched patches are stored at room temperature and protected from light.

All kit contents are kept within the final outer package until dispensing.

FIG. 6 (top-down view) is referenced for illustrating correct patch configuration on the scored release liner, and for documenting kit assembly steps ensuring patient usability, adherence, and safe handling during dispensing and disposal.

### Example 19: Real-World Patch Use and Handling Assessment

A real-world assessment was conducted to evaluate the usability, robustness, and effectiveness of the tizanidine transdermal drug delivery patch under typical end-user conditions.

### 1. User Application and Removal

Application Technique:

Subjects were instructed to apply the rectangular patch, measuring between 5 cm² and 20 cm² with rounded corners, directly to clean, dry skin. This is in direct accordance with the Instructions for Use provided in the kit. The scored release liner facilitated easy removal prior to application, as depicted in FIG. 6.
Adhesion and Wear:

Patches demonstrated consistent adhesion on various anatomical sites, maintaining tack for a minimum of 200 seconds at 6 grams pressure and shear adhesion over 100 grams (see product specifications in both provisional applications). Wear duration extended up to 48 hours without edge lift, peeling, or migration.
Wear Comfort and Discreteness:

The tan polyethylene-aluminum backing film provided an occlusive barrier, minimized water vapor transmission, and blended inconspicuously with skin, enhancing patient comfort and discretion throughout use.

### 2. Patch Integrity and Drug Delivery

Integrity After Wear:

Patches, after removal, retained structural integrity and uniform coat thickness (within ±10% of specification), confirming product robustness under typical storage, handling, and wear conditions.
Dissolution and Delivery Consistency:

Real-world use corroborated in vitro delivery profiles-cumulative drug release over 24-48 hours matched laboratory kinetics, with measured drug delivery of approximately 3 mg ±10% per patch (refer to FIG. 2A and FIG. 2B for representative dissolution timelines).
Minimal Residual Contamination:

Impurity profile remained <0.1% after wear, supporting clean handling and safe disposal, especially when individually-packaged with disposal pouches.

FIG. 6 clearly shows the patch's physical form and scoring for liner removal, supporting the study's findings on ease of user application and handling. FIGS. 2A and 2B further corroborate the match between real-world dissolution data and in vitro drug delivery profiles.

### Example 20: Physicochemical Properties and Testing Methods—Transdermal Tizanidine Patch

Each row contains a property name, the industry-standard or regulatory-aligned testing method, and an explicit specification or target. Table A provides significant material attributes, formulation parameters, manufacturing steps, or product performance metrics that are measurable and verifiable using established pharmaceutical analytical techniques. As such, the patch described herein can be robustly analyzed, e.g., for compliance with FDA/ICH requirements.

TABLE A

| Property | Testing Method | Specification |
|---|---|---|
| Drug content in patch (per | HPLC assay of extracted | 0.67-4 mg per patch |
| patch/batch) | patch samples | (proportional to area); ±5% |
| | | of label claim (per patch, |
| | | across batch) |
| Content uniformity across | HPLC/content assay, | ±5% of mean per patch and |
| patch/batch | gravimetric | batch |
| Particle size of tizanidine | Laser diffraction (D90) | <30 μm D90 |
| base | | |
| Matrix coat thickness | Micrometer (digital/feeler | 0.013″ ±10% (target), |
| | gauge) | measured at ≥3 points per |
| | | patch/roll |
| Coat weight per patch | Gravimetric (after | 0.045 g ±10% per 5 cm² |
| | liner/backing subtr.) | patch |
| Drug load in adhesive solids | HPLC analysis, weighing | 8-16% (w/w of dry matrix |
| | | solids) |
| Residual solvent content | GC per USP <467> | <500 ppm (for each process |
| | | solvent) |
| Total impurities (HPLC, | HPLC, ICH Q3A/Q3B | <0.1% by weight after |
| including degradants) | protocols | manufacture and after 12 |
| | | months at room |
| | | temperature/light-protected |
| Dissolution/release profile | USP apparatus, validated | Linear cumulative release; >80% |
| (in vitro) | buffer | cumulative delivery in |
| | | 48 hrs (3 mg ±10% per 48 hr, |
| | | 5 cm² patch, under neutral pH |
| | | buffe) |
| Skin permeation (ex vivo) | Franz diffusion cell | Cumulative flux matching |
| | (human skin or animal | target oral AUC; confirmed |
| | surrogate) | linearity over 24-48 hrs |
| Patch adhesion: | Peel test (90°), shear test | >200 sec (tack, 6 g); |
| peel/tack/shear | (grams), tack time | shear ≥100 g; 90° peel |
| | | 100-250 g |
| Moisture vapor transmission | ASTM E96 or modified | <1 g/m²/24 hr |
| rate (MVTR) (backing film) | | |
| Patch thickness (total, with | Micrometer | 0.008″ ±10% (entire patch) |
| liner/backing) | | |
| Uniformity of liner/backing | Manual/visual | No visible matrix |
| detachment | examination, peel force | detachment, clean peel, no |
| | | residue on patch after |
| | | removal |
| Patch stability (content, | HPLC, in vitro | Drug retention and impurity |
| delivery, impurities) | release/dissolution | levels within spec after 12 |
| | | months storage in sealed |
| | | pouch |
| Release liner specification | Vendor certificate, | 3M ScotchPak 9744 or 1022, |
| | micrometer | fluoropolymer-coated PET, |
| | | caliper 2.9 ± 0.02 mils |
| Backing film specification | Vendor certificate, | 3M ScotchPak 1012 (clear) |
| | MVTR testing | or 9738 (aluminized), MVTR |
| | | as above |
| Score line presence (release | Visual inspection | Scored liner extending ≥80% |
| liner) | | width, facilitates patch |
| | | removal without matrix |
| | | disruption |
| Occlusivity of patch | MVTR test | <1 g/m²/24 hr (as above), |
| | | confirms barrier |
| Individual patch packaging | Burst test, visual | No breach after sealing, |
| integrity | inspection | lot/date present |
| In-process thickness control | Micrometer, process | No segment deviates >10% |
| | control sheet | from mean |
| Drug/excipient homogeneity | HPLC mapping, visual, | No >5% local deviation in |
| in matrix | microscopic cross-section | drug content, no API |
| | | aggregates |
| Penetration enhancer content | HPLC, GC, or validated | 3-9% (w/w) of dry matrix |
| (e.g., PVP, DEG) | colorimetric | solids, each enhancer |
| Residual impurity | GC/USP <467> | <500 ppm, not detected by |
| (acetone/chloroform etc.) | | HPLC in final product |
| content | | |
| Stability (long-term, | ICH Q1A(R2); formal | Assay, total impurities, and |
| accelerated) | written protocol at 0, 1, 3, 6, | dissolution within product- |
| | 9, 12 months, with at least | specific acceptance. |
| | duplicate batches and at least | |
| | 3 replicate test units per time | |
| | point. | |

Although the foregoing description has been provided in connection with certain embodiments, it is to be understood that the invention is not limited to those embodiments. Various modifications and variations will be apparent to those skilled in the art in view of the above disclosure. The specification and examples are intended to be illustrative only, and the scope of the invention is defined by the claims appended hereto. It is contemplated that the appended claims will cover such modifications and embodiments as fall within the true spirit and scope of the invention.

1. A transdermal drug delivery patch comprising:
a monolithic adhesive matrix comprising an acrylic pressure-sensitive adhesive,
dispersed within the adhesive matrix, an effective amount of finely ground, micronized, or milled tizanidine base as the active pharmaceutical ingredient,
at least one permeation enhancer selected from povidone and diethylene glycol,
a solvent system comprising ethanol and/or ethyl acetate present during preparation of the adhesive matrix,
the monolithic adhesive matrix having a thickness of 0.013±0.004 inches,
a release liner comprising a fluoropolymer-coated polyester film in contact with the adhesive matrix, and
a backing film comprising a laminate including at least one polymeric film and a barrier layer,
wherein the patch has an area between 5 cm² and 20 cm² and is adapted to release tizanidine base transdermally over a period of at least 24 to 48 hours.

2. The transdermal patch of claim 1, wherein the adhesive comprises an acrylic copolymer having a —COOH or —OH functional group.

3. The transdermal patch of claim 1, wherein the tizanidine base present in the adhesive matrix is micronized to a particle size <30 microns D90 (by volume) as measured by laser diffraction, the tizanidine base being provided as a friable crystal or micronized material for enhanced dissolution and skin permeation, and the patch is configured such that tizanidine base content is uniform to within ±5% across the patch.

4. The transdermal patch of claim 1, wherein the patch is offered in a plurality of titratable sizes between 5 cm² and about 20 cm².

5. The transdermal patch of claim 1, wherein the drug load in the adhesive matrix comprises between about 8% and about 16% by weight of the solids in the adhesive layer, the drug content being uniform to within ±5% across individual patches and batches.

6. The transdermal patch of claim 1, wherein the adhesive matrix layer has a thickness of about 0.013 inches, ±10%.

7. The transdermal patch of claim 1, wherein said patch is manufactured and stored in a manner maintaining less than 0.1% total impurities as measured by HPLC for at least 12 months storage at ambient temperature and in darkness, with content uniformity and dissolution profile maintained throughout shelf life, and wherein tizanidine base is retained in a pharmaceutically active form, within the claimed impurity and uniformity limits, throughout the labeled shelf life.

8. The transdermal patch of claim 1, wherein a 5 cm² sample has a coat weight of about 0.045 grams, ±10%.

9. The transdermal patch of claim 1, further comprising both a polyvinylpyrrolidone and diethylene glycol as penetration enhancers dispersed in the adhesive.

10. The transdermal patch of claim 1, wherein the formulation contains <500 ppm acetone and <500 ppm chloroform as residual solvents.

11. The transdermal patch of claim 1, wherein the release liner comprises a fluoropolymer-coated polyester film having a caliper of 2.9±0.02 mils.

12. The transdermal patch of claim 1, wherein the backing film comprises a multi-layer laminate that includes a polymeric film and a metallic vapor barrier layer.

13. The transdermal patch of claim 1, wherein the patch is rectangular in shape with rounded corners.

14. The transdermal patch of claim 1, wherein the tizanidine base content varies by no more than ±5% across the patch.

15. The transdermal patch of claim 1, wherein the total related substance impurities are <0.1% by weight as measured by high-performance liquid chromatography, and tizanidine base content uniformity is maintained within ±5% across the patch and batch.

16. The transdermal patch of claim 1, wherein the patch delivers about 3 mg of tizanidine base, ±10%, over a period of 48 hours under neutral pH conditions, and achieves an in vitro or in vivo dissolution profile with linear cumulative delivery over 24-48 hours, the release profile matching or approximating the area under the concentration-time curve (AUC) of an equivalent oral dose without corresponding Cmax spikes or GI side effects, and wherein the patch maintains consistent release kinetics regardless of feeding state.

17. The transdermal patch of claim 1, comprising <500 ppm of ethyl acetate and <500 ppm of ethanol as residual solvents.

18. The transdermal patch of claim 1, wherein the release liner is provided with a score line extending across the patch to facilitate removal of the liner from the adhesive matrix.

19. The transdermal patch of claim 1, wherein the backing film is substantially occlusive, having a moisture vapor transmission rate (MVTR) of less than 1 g/m²/24 hr.

20. The transdermal patch of claim 1, wherein the adhesive matrix is produced by a process that includes in-process micrometer measurement of coating thickness, with coat thickness variation not exceeding±10% across the patch, and further includes sequential mixing of ingredients and immediate lamination to the backing film following drying, with no intermediate hold period.

21. The transdermal patch of claim 1, wherein the patch is packaged in a light-impermeable pouch to minimize photodegradation of the tizanidine base.

22. The transdermal patch of claim 1, wherein the patch remains stable with no increase in total impurities above 0.1% after storage at ambient temperature shielded from light for a period of at least 12 months, and shows no loss in therapeutic performance over its labeled shelf life.

23. The transdermal patch of claim 1, wherein the patch is produced in a plurality of discrete sizes between 5 cm² and 20 cm² to provide titratable dosing.

24. The transdermal patch of claim 1, wherein the release liner is optionally selected from 3M ScotchPak 9744 or ScotchPak 1022 fluoropolymer-coated polyester films.

25. The transdermal patch of claim 1, wherein the preparation of the adhesive matrix comprises the sequential addition of ethanol, povidone, diethylene glycol, tizanidine base, and, finally, adhesive.

26. The transdermal patch of claim 1, wherein, during manufacture, the coating is laminated to the backing film immediately after drying without any hold time.

27. The transdermal patch of claim 1, wherein the tizanidine base is dispersed as a particulate suspension within the solvent-based adhesive formulation prior to coating and drying.

28. The transdermal patch of claim 1, wherein the patch, when used as intended, achieves a steady-state plasma tizanidine base level with a Cmax lower than that of an equivalent orally administered dose, and delivers tizanidine base with a reduced incidence of gastrointestinal side effects compared to oral administration, including in patients sensitive or intolerant to oral therapy, or in those exhibiting pharmacokinetic variability with oral administration.

29. A method of treating increased muscle tone associated with spasticity in a subject in need thereof, comprising transdermally administering tizanidine base by applying to the skin the transdermal drug delivery patch of claim 1, wherein said patch is adapted to deliver an effective amount of tizanidine base over a period of at least 24 to 48 hours to reduce muscle tone and/or alleviate the symptoms of spasticity, in patients who are unable or unwilling to swallow orally.

30. A kit for transdermal administration of tizanidine base, comprising:
at least one transdermal patch of claim 1;
wherein the patch, when applied as directed, is configured to deliver tizanidine base with a reduced incidence of gastrointestinal side effects and with steady-state plasma levels compared to oral administration;
and, separately packaged with the at least one patch, instructions for use directing application of the patch to the skin to treat increased muscle tone associated with spasticity, provide titratable dosing, and maintain reduced gastrointestinal side effects relative to oral tizanidine therapy;
and further comprising at least one additional component selected from: (a) individually sealed pouches for each patch, (b) a child-resistant package, (c) a calendar or dosing schedule aid, (d) a disposal pouch for used patches, (e) a titration card or dose adjustment guide, (f) a desiccant or moisture-absorbing element, (g) a temperature indicator, (h) a QR code or web address for digital instructions, (i) multilingual instructions, and (j) a sterile wipe or pad for skin preparation.
